(19) United States
(12) Patent Application Publication
(10) Pub. No.: US 2003/0143561 A1
(43) Pub. Date: Jul. 31, 2003

(54) Nucleoside derivatives for library preparation

(75) Inventors: Henrik Pedersen, Bagsvaerd (DK); Christian Klarner Sams, Frederiksberg C (DK); Frank Abilgaard Slok, Kobenhavn N (DK); Lene Hyldtoft, Virum (DK); Michael Anders Godskesen, Vedbaek (DK)

Correspondence Address: BROWDY AND NEIMARK, P.L.L.C., 624 NINTH STREET, NW, SUITE 300, WASHINGTON, DC, 20001-5303, US

(73) Assignee: Nuevolution A/S, Copenhagen (DK)

(21) Appl. No.: 10/175,500

(22) Filed: Jun. 20, 2002

Related U.S. Application Data
(60) Provisional application No. 60/299,443, filed on Jun. 21, 2001.

(30) Foreign Application Priority Data
Jun. 20, 2001 (DK) PA 2001 00962

(51) Int. Cl. C12Q 1/68; C07H 21/04; C07H 19/04; C07D 413/14; C07D 473/34; C07D 473/22; C07K 14/00

(52) U.S. Cl. 435/6; 530/350; 536/23.1; 536/26.1; 544/118; 544/233; 544/276; 544/277

(57) ABSTRACT

Nucleoside derivatives as building blocks for templated libraries are described.

### TECHNICAL FIELD OF THE INVENTION

The present invention relates to nucleotide derivatives. The nucleotide derivatives of the present invention are useful in the preparation of templated molecules.

### BACKGROUND

The generation of molecules carrying new properties remains a challenging task. Recently, a number of procedures have been suggested that should allow a more efficient generation and screening of a larger number of molecules. The approaches taken involve the encoding and/or templating of molecules other than natural biopolymers such as peptide, RNA and DNA. These approaches allow the researcher to generate and screen a huge number of molecules in a short time. This should lead to better molecules carrying the desired properties.

The central dogma of biology describes the one-way flow of information from DNA to RNA to protein. Recently, methods such as phage display, peptides-on-plasmids, ribosome display and mRNA-protein fusion have been developed, allowing the transfer of information from the level of protein/peptide to RNA or DNA. This has enabled the use of molecular evolution to be applied on huge numbers of peptides that are exposed to an enrichment process, where after the enriched pool of molecules (enriched for a particular feature, such as binding to receptor protein) are amplified, by exploiting information flow from the peptide to DNA and then amplifying the DNA.

More recently, approaches have been developed that allow the encoding of polypeptides and other biochemical polymers. An example of this approach is disclosed in U.S. Pat. No. 5,723,598, which pertains to the identification of a biochemical polymer that participates in a preselected binding interaction with a target to form a binding reaction complex. The prior art method encompasses the generation of a library of bifunctional molecules. One part of the bifunctional molecule is the biochemical polymer and the other part is an identifier oligonucleotide comprising a sequence of nucleotides which encodes and identifies the biochemical polymer. Following the generation of the library of the bifunctional molecules, a partitioning with respect to affinity towards the target is conducted and the identifier oligonucleotide part of the bi-functional molecule is amplified by means of PCR. Eventually, the PCR amplicons are sequenced and decoded for identification of the biochemical polymer. This approach does not, however, allow one-pot amplification of the library members. Thus the flow of information from the identifier sequence to the biochemical polymer is restrained.

Halpin and Harbury have in WO 00/23458 suggested an improvement to the approach stipulated immediately above, wherein the molecules formed are not only identified but also directed by the nucleic acid tag. The approach is based on the traditional split-and-combine strategy for synthesis of combinatorial libraries comprising two or more synthetic steps. Plurality nucleic acid templates are used, each having at one end a chemical reactive site and dispersed throughout the strand a plurality of codons regions, each of said codon regions in turn specifying different codons. Separately, each of the strands, identified by a first codon region, is reacted at the chemical reaction sites with specific selected reagents. Subsequently, all the strands are pooled and subjected to a second partitioning based on a second codon region. The split-and-combine method is conducted an appropriate number of times to produce a library of typically between 10³ and 10⁶ different compounds. The split-and-combine method is cumbersome and generates only a relatively small library.

The various known methods for production of libraries as well as novel not yet public methods of the present applicant require building blocks comprising a complementing element able to recognize a coding element of a template. The present invention aims at providing such building blocks. In one aspect, the present invention relates to building blocks capable of being incorporated by a polymerase or reverse transcriptase. In another aspect, the present invention relates to building blocks capable of being incorporated in the absence of an enzyme. The building block comprises, apart from the complementing element, a linker and a functional entity. The functional entity of the compounds of the present invention may comprise an amino acid precursor. When a plurality of the building blocks are incorporated into a complementing template the functional entities are able to be linked to each other, thus forming a templated molecule, the synthesis of which is directed by the coding elements of the template. The characteristic alkynylene moiety of the linkers of the present invention makes it possible to display the functional entity in the major groove of a double stranded molecule. When two or more functional entities are displayed simultaneously in the major groove reactive groups of the functional enti-simultaneously in the major groove reactive groups of the functional entities may react, either directly or via a suitable bridging molecule, to form a linkage between the functional entities. Thus, upon proper incorporation of a plurality of the compounds of the invention it is possible to form a templated molecule by linking each of the functional entities. The linkers may optionally be cleaved simultaneously with or after the formation of the templated molecule. Preferably at least one linker remains uncleaved to attach the templated molecule to the template which templated the synthesis thereof or a complementing template. A library of different complexes of template (or complementing template) and templated molecule may be subjected to various screening methods, such as affinity screening, known to the person skilled in the art to identify one or more templated molecule with the desired effect.

The compounds of the present invention may be used for the production of natural α-peptides. However, recently a strong interest has been observed in academic societies for peptides other than α-peptides, such as β-peptides, γ-peptides, and δ-peptides. In one aspect of the invention it is contemplated to provide building blocks for the formation of molecules based on such artificial peptides.

### SUMMARY OF THE INVENTION

The present invention relates to nucleoside derivatives of the general formula:

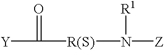

Wherein Y is a group

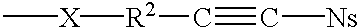

Wherein:

X is a hetero atom selected from the group O, S, Se or a group NR⁴, wherein R⁴ is hydrogen or an optionally substituted linear or branched C₁₋₆ alkyl or C₂₋₆ alkenyl. R² is selected from the group consisting of C₁₋₆ alkylen, C₂₋₆ alkylenylen, C₂₋₆ alkynylen, C₃₋₆ cycloalkylen, heterocycloalkylen, —CH₂—O—, arylen or heteroarylen, wherein each of the groups R² are substituted with 0-3 R⁸ groups independently selected from ═O, ═S, —F, —Cl, —Br, —I, —OCH₃, —NO₂ or C₁₋₆ alkyl, and Ns is a nucleoside analogue consisting of a nucleobase and a backbone unit, or Y is —OR³, wherein R³ is H or an acid protective group

R(S) is a C₁₋₄ alkylen, C₃₋₁₀ cycloalkylen, aryl, heterocycloalkyl or heteroaryl substituted by n sidechains S, wherein n is an integer of 0 to 4

R¹ is H, C₁₋₆ alkyl substituted with 0-3 R⁹ where R⁹ is independently selected from ═O, Cl, Br, —CN, —OR⁶, —SR⁶, —NR⁶R⁷, —COOR⁶, —CONR⁶R⁷, —SO₂NR⁶R⁷ or a C₁₋₆ alkylen group forming a ringstructure with S

R⁶ and R⁷ are independently selected from H, C₁₋₆ linear alkyl, C₁₋₆ branched alkyl, C₁₋₆ cycloalkyl, aryl, heteroaryl, aralkyl, or hetero aralkyl.

S is C₁₋₆ linear alkyl, C₃₋₆ branched alkyl, C₃₋₆ cycloalkyl, aryl, heteroaryl, aralkyl, hetero aralkyl substituted with 0-3 R⁵ where R⁵ is independently selected from ═O, Cl, Br, —CN, —OR⁶, —SR⁶, —NR⁶R⁷, —COOR⁶, —CONR⁶R⁷, —SO₂NR⁶R⁷.

Z is H, an amino protective group or a group

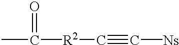

with the proviso, that when Y is not

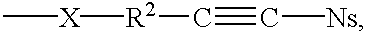

Z is

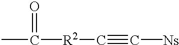

Such derivatives enable the preparation of large libraries of compounds templated by nucleic acids or analogues thereof. In particular, the present invention relates to building blocks carrying amino acid components allowing the construction of oligopeptides containing natural- as well as unnatural amino acid fragments.

In a preferred embodiment the alkynylen linker is connected to the nucleobase of a nucleoside analogue.

In another preferred embodiment the alkynylen linker is connected to the nucleobase of a nucleoside analogue in the 7 position of the bicyclic purine nucleobases and the 5 position of the monocyclic pyrimidine bases which ensures the positioning of the functional entity into the major groove of the nascent oligomer-complex.

The combination of R² and X determines the stability of the linkage between the functional entity and the complementing element. Hence different R²—X combinations require different cleavage conditions allowing some linkers to be cleaved while others remain intact.

In a preferred embodiment R² is selected from the group consisting of C₁₋₆ alkylen, C₂₋₆ alkylenylen, C₂₋₆ alkynylen, heterocycloalkylen, —CH₂—O—, arylen or heteroarylen, each of the groups R² are substituted with 0-3 R⁸ groups independently selected from ═O, —F, —Cl, —Br, —NO₂, C₁₋₆ alkyl.

In a preferred embodiment R² is selected from the group consisting of C₁₋₆ alkylen, C₂₋₆ alkynylen, heterocycloalkylen, —CH₂—O—, arylen or heteroarylen, each of the groups R² are substituted with 0-2 R⁸ groups independently selected from ═O, —F, —NO₂, C₁₋₆ alkyl.

In a preferred embodiment R² is selected from the group consisting of —CH₂—, —CH₂CH₂—,

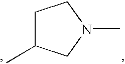

—CH₂—O—, or arylen each of the groups R² are substituted with 0-2 R⁸ groups independently selected from ═O, —F, —NO₂, C₁₋₆ alkyl.

In a preferred embodiment R² is selected from the group consisting of —CH₂—, —CH₂CH₂—,

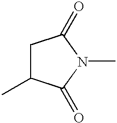

—CH₂—O—, or arylen.

In a preferred embodiment R² is selected from the group consisting of —CH₂—, —CH₂CH₂—,

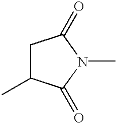

or arylen.

In a preferred embodiment X is O

In a preferred embodiment X is S

In a preferred embodiment X is NR⁴

In a preferred embodiment X is NR⁴ and R⁴ is H or —CH₃

In a preferred embodiment X is NH

In a preferred embodiment R(S) is a C₁₋₄ alkylene, C₃₋₁₀ cycloalkylen, aryl, heterocycloalkyl or heteroaryl substituted by n sidechains S, wherein n is an integer of 0 to 3

In a preferred embodiment R(S) is a C₁₋₄ alkylene, aryl or heteroaryl substituted by n sidechains S, wherein n is an integer of 0 to 3

In a preferred embodiment R(S) is a C₁₋₄ alkylene substituted by n sidechains S, wherein n is an integer of 0 to 3

In a preferred embodiment R(S) is a C₁₋₂ alkylene substituted by n sidechains S, wherein n is an integer of 0 to 3

In a preferred embodiment R(S) is a C₁₋₂ alkylene substituted by n sidechains S, wherein n is an integer of 0 to 2

In a preferred embodiment R(S) is a C₁₋₂ alkylene substituted by n sidechains S, wherein n is an integer of 0 to 1

In a preferred embodiment S is C₁₋₆ linear alkyl, C₃₋₆ branched alkyl, C₃₋₆ cycloalkyl, aryl, heteroaryl, aralkyl, hetero aralkyl substituted with 0-3 R⁵ where R⁵ is independently selected from ═O, Cl, Br, —CN, —OR⁶, —SR⁶, —NR⁶R⁷, —COOR⁶, —CON R⁶R⁷—SO₂NR⁶R⁷ where R⁶ and R⁷ are independently selected from H, C₁₋₃ linear alkyl, C₃₋₆ cycloalkyl, aryl, heteroaryl, aralkyl, or hetero aralkyl.

In a preferred embodiment S is C₁₋₆ linear alkyl, C₃₋₆ branched alkyl, C₃₋₆ cycloalkyl, aryl, heteroaryl, aralkyl, hetero aralkyl substituted with 0-2 R⁵ where R⁵ is independently selected from ═O, Cl, —CN, —OR⁶, —SR⁶, —NR⁶R⁷, —COOR⁶, —CONR⁶R⁷, SO₂NR⁶R⁷ where R⁶ and R⁷ are independently selected from H, C₁₋₃ linear alkyl, aryl, heteroaryl, aralkyl, or hetero aralkyl.

In a preferred embodiment S is C₁₋₆ linear alkyl, C₃₋₆ branched alkyl, C₃₋₆ cycloalkyl, aryl, heteroaryl, aralkyl, hetero aralkyl substituted with 0-2 R⁵ where R⁵ is independently selected from ═O, Cl, —CN, —OR⁶, —SR⁶, —NR⁶R⁷, —COOR⁶, —CONR⁶R⁷—SO₂NR⁶R⁷ where R⁶ and R⁷ are independently selected from H and C₁₋₃ linear alkyl

In a preferred embodiment S is C₁₋₆ linear alkyl, C₃₋₆ branched alkyl, C₃₋₆ cycloalkyl, aryl, heteroaryl, aralkyl, hetero aralkyl substituted with 0-1 R⁵ where R⁵ is selected from ═O, Cl, —CN, —OR⁶, —SR⁶, —NR⁶R⁷, —COOR⁶, —CONR⁶R⁷, —SO₂NR⁶R⁷ where R⁶ and R⁷ are independently selected from H and C₁₋₃ linear alkyl

In a preferred embodiment S is C₁₋₆ linear alkyl or aryl substituted with 0-1 R⁵ where R⁵ is selected from ═O, Cl, —CN, —OR⁶, —SR⁶, —NR⁶R⁷, —COOR⁶, —CONR⁶R⁷—SO₂NR⁶R⁷ where R⁶ and R⁷ are independently selected from H and C₁₋₃ linear alkyl

In a preferred embodiment S is C₁₋₆ linear alkyl or aryl.

In a preferred embodiment R¹ is H, C₁₋₆ alkyl substituted with 0-1 R⁹ where R⁹ is independently selected from ═O, Cl, Br, —CN, —OR⁶, —SR⁶, —NR⁶R⁷, —COOR⁶, —CONR⁶R⁷, —SO₂NR⁶R⁷, R⁶ and R⁷ are independently selected from H, C₁₆ linear alkyl, C₁₋₆ branched alkyl, C₁₋₆ cycloalkyl, aryl, heteroaryl, aralkyl, or hetero aralkyl, or a C₁₋₆ alkylen group forming a ringstructure with S.

In a preferred embodiment R¹ is H, C₁₋₆ alkyl or a C₁₋₆ alkylen group forming a ringstructure with S

In a preferred embodiment R¹ is H or a C₁₋₆ alkylen group forming a ringstructure with S.

In a preferred embodiment R¹ is H or C₁₋₆ alkyl.

In a preferred embodiment R¹ is H.

In a preferred embodiment Z is H, an amino protective group selected from the group of formyl, acetyl, trifluoroacetyl, benzoyl, tert-butyloxycarbonyl, triphenylmethyl, benzyl, benzyloxycarbonyl or tosyl or a group

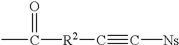

with the proviso, that when Y is not

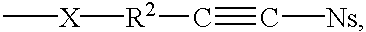

then Z is

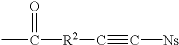

In a preferred embodiment Z is H, an amino protective group selected from the group of acetyl, trifluoroacetyl, tert-butyloxycarbonyl or tosyl or a group

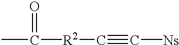

with the proviso, that when Y is not X—R²—C≡C—Ns then Z is

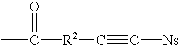

In a preferred embodiment the nucleobase is uracil or cytosine modified in the 5 position or 7-adeazaadenine or 7-deazaguanidine modified in the 7 position.

In a preferred embodiment the backbone unit type is DNA, RNA, Oxy-LNA, Thio-LNA, Amino-LNA, Phosphorthioate, 2′-O-methyl, PNA or Morpholino as described in chart 3.

In a preferred embodiment the backbone unit type is DNA, RNA, Oxy-LNA, PNA or Morpholino

In a preferred embodiment the backbone unit type is DNA, PNA or Oxy-LNA

In a preferred embodiment the backbone unit type is DNA

In a preferred embodiment the backbone unit type is Oxy-LNA

In a preferred embodiment the backbone unit type is PNA

Using di- or trimeric building blocks results in improved recognition of the nucleobases on the template, especially when chemical methods are used to oligomerise the nucleoside analogues. (Schmidt; 1997; Nucleic Acids Research; 4792-4796) The use of oligomeric nucleoside analogues allow the direct annealing of building blocks to the template without the need for chemical- or enzymatic incorporation. In a preferred embodiment more nucleoside analogues are connected via their backbone structures forming di-, tri- or oligomeric nucleoside analogues as building blocks

In a preferred embodiment Y is

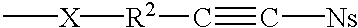

or —OR³ wherein R³ is selected from the group H, C₁₋₃ alkyl, allyl, benzyl, tert-butyl or triphenylmethyl.

Aralkyl is an aryl connected to a C₁₋₆ alkylene

Complementing element recognizes combinations of nucleobases in the template and consists of at least one nucleoside analogue, optionally attached to a series of at least one backbone unit carrying a nucleobase.

Complex is a templated molecule linked to the template that templated the synthesis of the templated molecule. The template can be a complementing template as defined herein that is optionally hybridised or otherwise attached to a corresponding template of linked coding elements.

Heteroaryl designates an unsaturated cyclic structure consisting of 2-5 carbon atoms and 1-3 heteroatoms selected from O, S, N or P.

Heterocycloalkyl designates a saturated or partially saturated cyclic structure consisting of 2-5 carbon atoms and 1-3 heteroatoms selected from O, S, N or P.

Library is in this context a collection of molecules.

Nucleoside analogue is any combination of a nucleobase and a backbone unit.

Abbreviations

| | |
|---|---|
| DCC | N,N′-Dicyclohexylcarbodiimide |
| DIC | Diisopropylcarbodiimide |
| DIEA | Diethylisopropylamin |
| DMAP | 4-Dimethylaminopyridine |
| EDC | 1-Ethyl-3-(3′-dimethylaminopropyl)carbodiimide-HCl |
| HATU | 2-(1H-7-Azabenzotriazole-1-yl)-1,1,3,3- |
| | tetramethyluronium hexafluorophosphate |
| HBTU | 2-(1H-Benzotriazole-1-yl)-1,1,3,3-tetramethyluronium |
| | hexafluorophosphate |
| HOAt | N-Hydroxy-7-azabenzotriazole |
| HOBt | N-Hydroxybenzotriazole |
| NHS | N-hydroxysuccinimid |
| PyBoP | Benzotriazole-1-yl-oxy-tris-pyrrolidino-phosphonium |
| | hexafluorophosphate |
| PyBroP | Bromo-tris-pyrrolidino-phosphonium hexafluorophos- |
| | phate |
| TBTU | 2-(1H-Benzotriazole-1-yl)-1,1,3,3-tetramethyluronium |
| | tetrafluoroborate |
| TEA | Triethylamine |

### BRIEF DESCRIPTION OF THE CHARTS

In chemical structure drawings throughout the document, hydrogen atoms on terminal carbon atoms are not explicitly shown.

### DETAILED DESCRIPTION OF THE INVENTION

Building blocks consist apart from a linker and a functional entity of one or more nucleoside analogues i.e. pairs of nucleobases and backbone units forming the complementing entity and may as such be considered a nucleoside derivative. The nucleobase may be of natural or of synthetic origin but all shares the common feature of being able to selectively recognize one other nucleobase. Examples of such base pairs are shown in chart 1

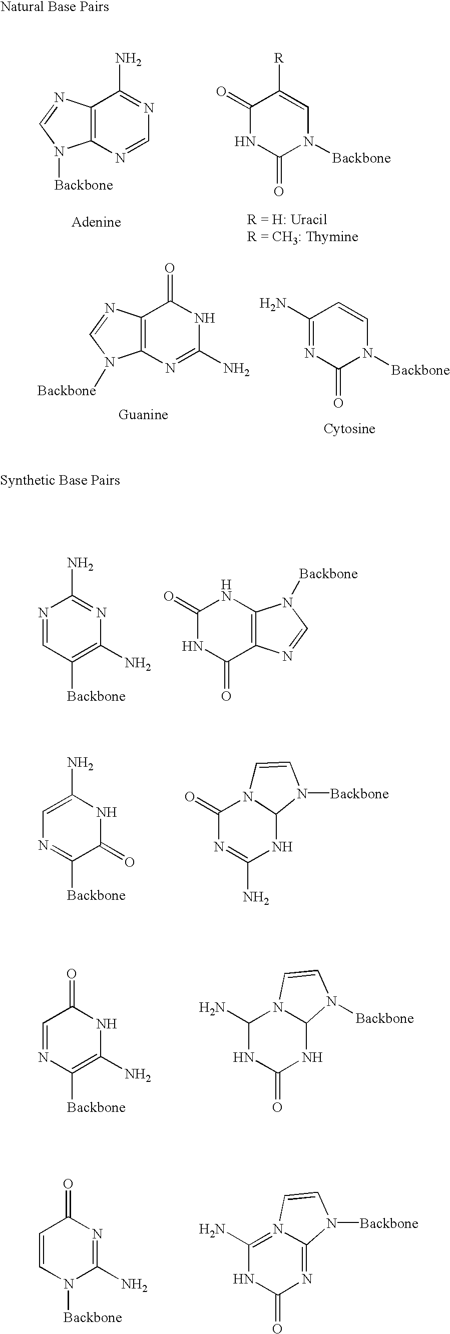

Chart 1 Natural and Synthetic nucleobases.

Also, modifications to both natural- and synthetic nucleobases is possible without obliteration of the mutual recognition properties, e.g. replacing the N-7 atom of adenine or guanidine with a carbon atom affords 7-deaza adenine and 7-deaza guanine respectively (Chart 2) that still recognises natural thymine or uracil and cytosine, respectively. Further the introduction of substituents at certain positions on the complementing entity is also possible.

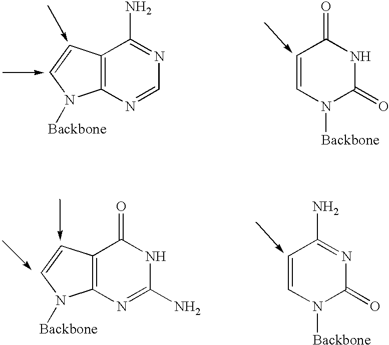

Chart 2. 7-deaza-adenine, uracil, 7-deaza-guanidine and cytosine. Arrows indicate preferred sites of substitution on the nucleobase of the complementing entity that direct the functional entity into the major groove of the nascent oligomer complex.

The backbone units of the building blocks may contain a set of reactive groups that enables enzymatic or chemical oligomerisation of the building blocks. Examples of backbone unit structures are given in chart 3

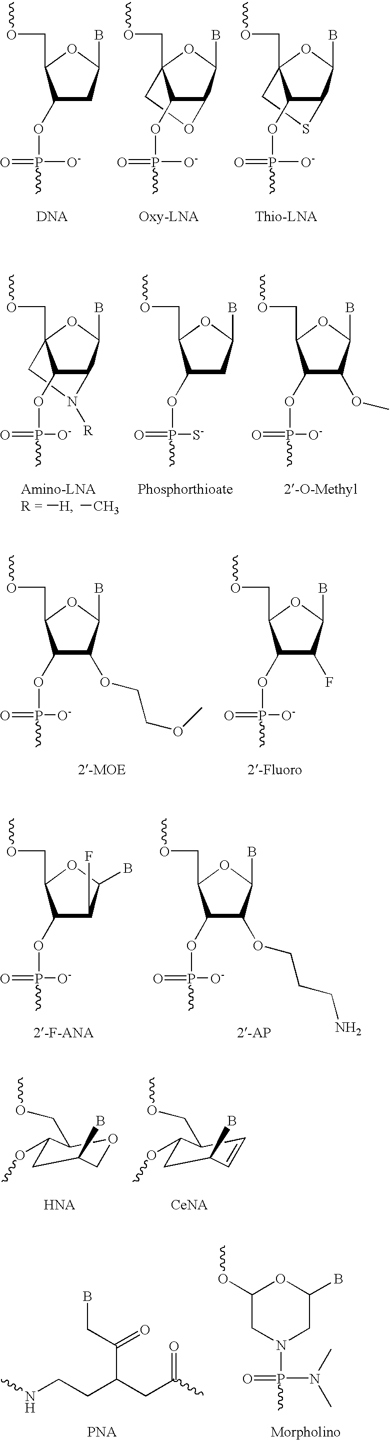

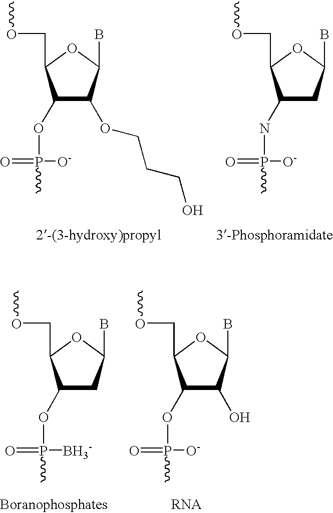

Chart 3 Backbone units used and building blocks. B designates the nucleobase and wavy bonds show points of oligomerisation.

Building blocks may be oligomerised using enzymatic or chemical methods. (Schmidt; 1997; Nucleic Acids Research; 4792-4796, Inoue; 1984; Journal of Molecular Biology, 669-676, Schmidt; 1997; Nucleic Acids Research; 4797-4802) Enzymatic incorporation is typically based on the use of 5′-O-triphosphate building blocks with a ribose derived backbone unit. Chemical incorporation of building blocks with a ribose derived backbone unit relies on the use of an activated phosphate ester e.g. a phoshporimidate. (Zhao; 1998; J. Org. Chem.; 7568-7572) For peptide backbone units, peptide coupling reagents are employed. As shown in chart 3 several modifications of the natural DNA- and RNA backbone is possible, particularly the 2-position of the ribose entity is well suited for functional entity linkage.

The linker is based on a rigid alkynylene spacer that positions the functional entity away from the back bone of the oligomer complex:

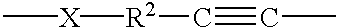

X is a hetero atom selected from the group O, S, Se or a group NR⁴, wherein R⁴ is hydrogen or an optionally substituted linear or branched C₁₋₆ alkyl or C₂₋₆ alkenyl. R² is selected from the group consisting of C₁₋₆ alkylen, C₂₋₆ alkylenylen, C₂₆ alkynylen, C₃₋₆ cycloalkylen, heterocycloalkylen, —CH₂—O—, arylen or heteroarylen, wherein each of the groups R² are substituted with 0-3 R⁸ groups independently selected from ═O, ═S, —F, —Cl, —Br, —I, —OCH₃, —NO₂ or C₁₋₆ alkyl

The functional entity is an aminoacid derivative:

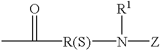

Wherein:

R(S) is a C₁₋₄ alkylen, C₃₋₁₀ cycloalkylen, aryl, heterocycloalkyl or heteroaryl substituted by n sidechains S, wherein n is an integer of 0 to 4

R¹ is H, C₁₋₆ alkyl substituted with 0-3 R⁹ where R⁹ is independently selected from ═O, Cl, Br, —CN, —OR⁶—SR⁶—NR⁶R⁷, —COOR⁶, —CONR⁶R⁷, —SO₂NR⁶R⁷ or a C₁₋₆ alkylen group forming a ringstructure with S

R⁶ and R⁷ are independently selected from H, C₁₋₆ linear alkyl, C₁₋₆ branched alkyl, C₁₋₆ cycloalkyl, aryl, heteroaryl, aralkyl, or hetero aralkyl.

S is C₁₋₆ linear alkyl, C₃₋₆ branched alkyl, C₃₋₆ cycloalkyl, aryl, heteroaryl, aralkyl, hetero aralkyl substituted with 0-3 R⁵ where R⁵ is independently selected from ═O, Cl, Br, —CN, —OR⁶—SR⁶, —NR⁶R⁷, —COOR⁶, —CONR⁶R⁷, —SO₂NR⁶R⁷.

Z is H, an amino protective group

General Synthesis Procedures

The compounds of the invention are generally prepared by two different methods.

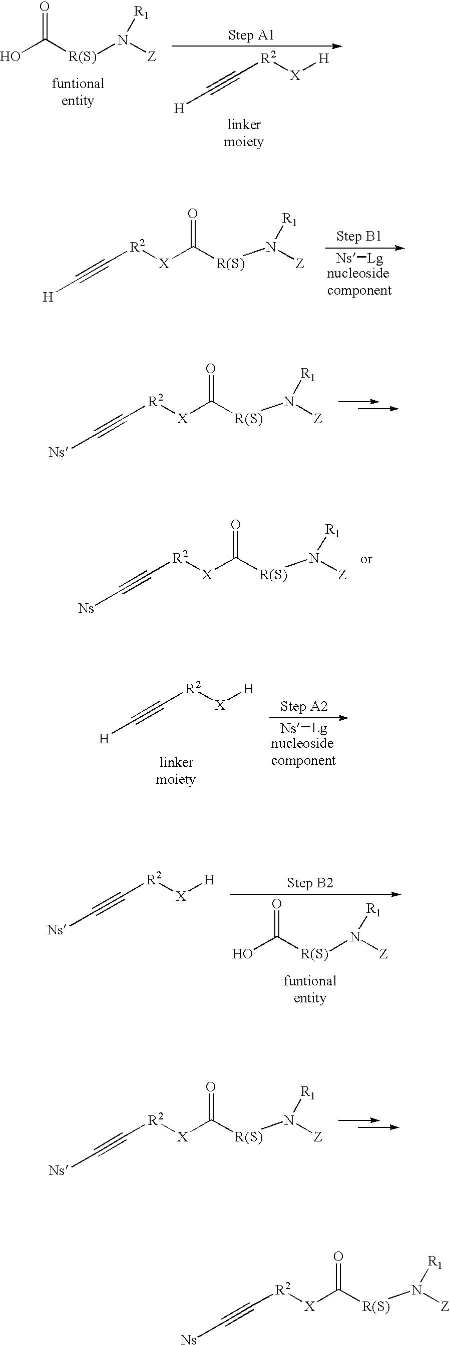

Ns′ is a precursor of Ns, e.g. a 3′-O-5′-O-protected nucleoside.

Lg is a leaving group suitable for Sonogashira couplings exemplified by but not limited to Br and 1.

Step A1

The amino acid derivative (functional entity) (10.37 mmol) is dissolved in a solvent exemplified by but not limited to dichloromethane, 1,2-dichloroethane, 1,2-dichloropropane, tetrahydrofuran, dimethylformamid or a mixture hereof and added a peptide coupling reagent (12.44 mmol, 1.2 eq) exemplified by but not limited to EDC, DCC, DIC, HATU, HBTU, PyBoP or PyBroP optionally in the presence of a peptide coupling enhancer like HOBt, HOAt, or NHS at a temperature of −20-100° C. preferably 0-50° C. To this mixture, the linker moiety (15.55 mmol, 1.5 equiv) is added optionally in the presence of DMAP (1.04 mmol, 0.1 eq) and the reaction is left 2-16 h. Upon evaporation of volatiles, the residue is taken up in dichloromethan and washed with HCl (aq, 0.1 M); NaHCO₃ (aq, sat); and water. Removal of dichloromethan affords the crude product which is further purified by chromatography if necessary.

Step B1

A solution of the nucleoside component (0.34 mmol) in a solvent like dimethylformamid, dimethylsulfoxid, toluene, tetrahydrofuran, water, ethanol, methanol or a mixture herof is added a terminal alkyne (the linker moiety-funtional entity) (0.69 mmol, 2 eq) and a base like DIEA (0.25 mL) and is purged with Ar for 5 min. Tetrakis triphenylphosphine palladium (0.03 mmol, 0.1 eq) and CuI (0.07 mmol, 0.2 eq) is added and the reaction is run at 20-100° C., preferably at 20-50° C., and kept there for 20 h. Evaporation of volatiles followed by chromatography affords the desired modified nucleoside.

Step A2

A solution of the complementing element precursor (0.34 mmol) in a solvent like dimethylformamid, dimethylsulfoxid, toluene, tetrahydrofuran, water, ethanol, methanol or a mixture herof is added a terminal alkyne (the linker moiety) (0.69 mmol, 2 eq) and a base like DIEA (0.25 mL) and is purged with Ar for 5 min. Tetrakis triphenylphosphine palladium (0.03 mmol, 0.1 eq) and CuI (0.07 mmol, 0.2 eq) is added and the reaction is run at 20-100° C., preferably at 20-50° C., and kept there for 20 h. Evaporation of volatiles followed by chromatography affords the desired modified nucleoside.

Depending on the nature of Ns′ several steps known from literature may be required to convert Ns′ into Ns e.g. Protective group removal (Greene; 1999;) or conversion of 5′OH groups of nucleosides into 5′O-triphosphates or phosphorimidazolides.(Zhao; 1998; J. Org. Chem.; 7568-7572)

Nucleoside analogues with phosphate linkages in the backbone may be combined with wild type nucleotides to form di-, tri- or oligomeric buildingblocks. Likewise, nucleoside analogues having a PNA backbone unit may be combined with PNA monomers to form di-, tri- or oligomeric building blocks.

Step B2

The amino acid derivative (functional entity) (10.37 mmol) is dissolved in a solvent exemplified by but not limited to dichloromethane, 1,2-dichloroethane, 1,2-dichloropropane, tetrahydrofuran, dimethylformamid or a mixture hereof and added a peptide coupling reagent (12.44 mmol, 1.2 eq) exemplified by but not limited to EDC, DCC, DIC, HATU, HBTU, PyBoP or PyBroP optionally in the presence of a peptide coupling enhancer like HOBt, HOAt, or NHS at a temperature of −20-100° C. preferably 0-50° C. To this mixture, the linker-nucleoside component (15.55 mmol, 1.5 equiv) obtained in step A2 is added optionally in the presence of DMAP (1.04 mmol, 0.1 eq) and the reaction is left 2-16 h. Upon evaporation of volatiles, the residue is taken up in dichloromethan and washed with HCl (aq, 0.1 M); NaHCO₃ (aq, sat); and water. Removal of dichloromethan affords the crude product which may be further purified by chromatography if necessary.

Depending on the nature of Ns′ several steps known from literature may be required to convert Ns′ into Ns e.g. protective group removal, conversion of 5′-OH groups of ribose derived backbone units into 5′-O-triphosphates or phosphorimidazolides. (Zhao; 1998; J. Org. Chem.; 7568-7572). For peptide derived backbone units other types of modifications are required. (Hyrup; 1996; Bioorganic & medicinal chemistry, 5-23)

Nucleoside analogues carrying a ribose derived backbone unit may be combined with wild type nucleotides to form di-, tri- or oligo-nucleotid building blocks. Likewise, nucleoside analogues having a peptide backbone unit may be combined with PNA monomers to form di-, tri or oligo peptidic building blocks.

### EXAMPLES

### Example 1 to 7

### Preparation of the Mononucleotide Building Block (I)

Building block I may be prepared according to the general scheme shown below:

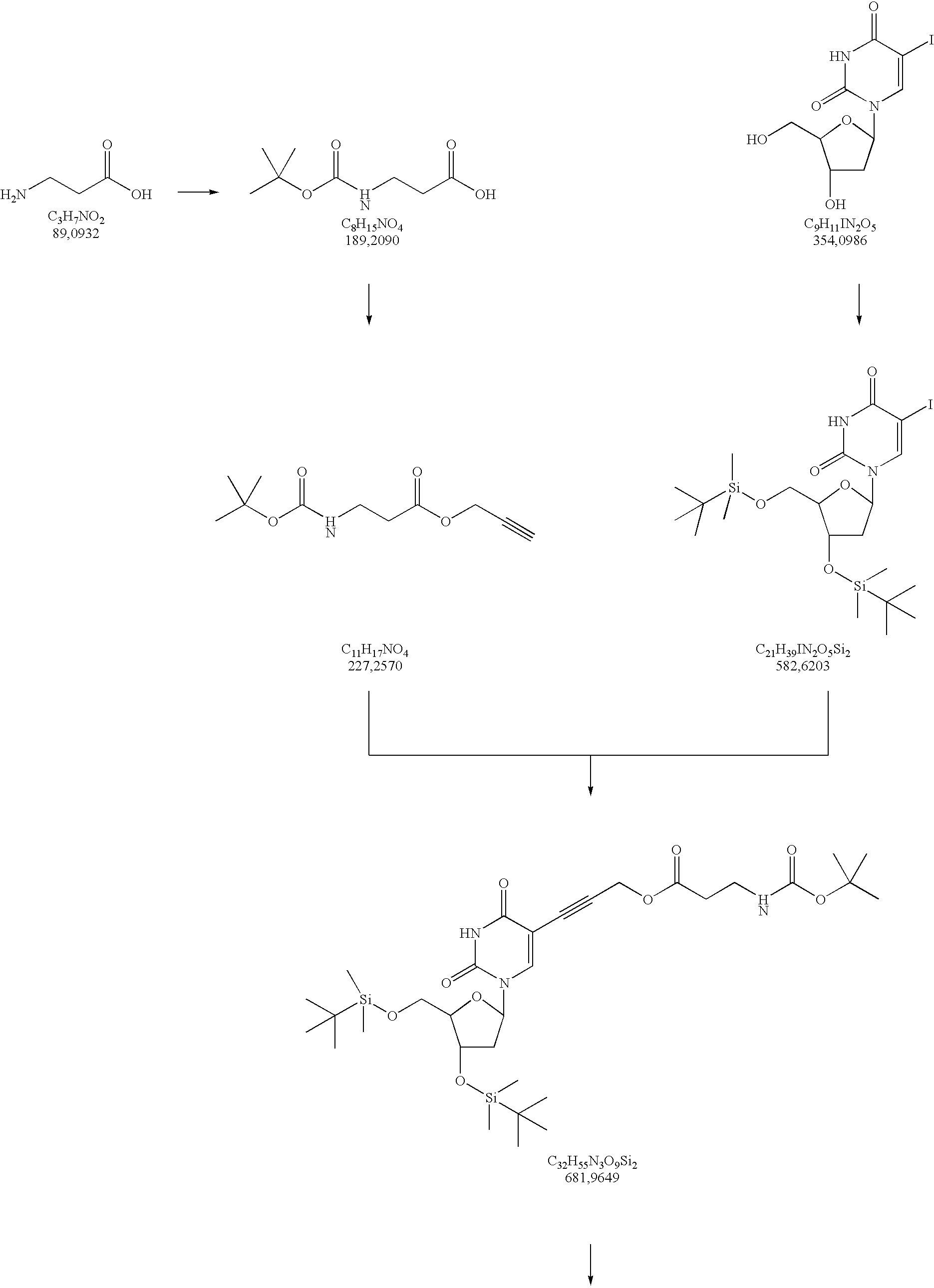

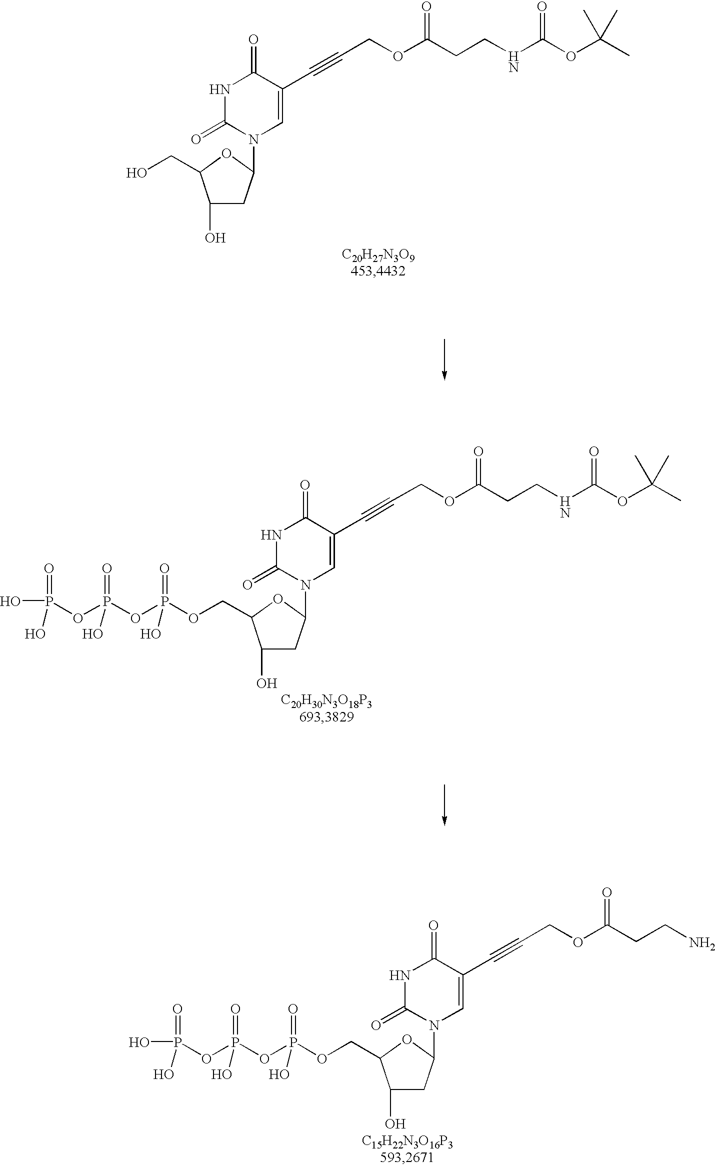

### Example 1

### Preparation of 3-tert-Butoxycarbonylamino-propionic acid (N-Boc-β-alanine)(1 a)

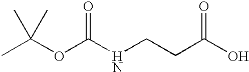

To a solution of β-alanine (2,25 g, 25 mmol) in aq. NaHCO₃ (25 mL) were added di-tert-butyl dicarbonate (4,36 g, 20 mmol) and acetonitrile (25 mL). The reaction mixture was stirred at room temperature for 18 h. EtOAc (100 mL) was added and pH was adjusted to 4-5 by addition of NaH₂PO₄. The product was extracted into EtOAc (3×50 mL), dried (Na₂SO₄), and evaporated to dryness under vacuum to afford 3.71 g (98%)

¹H NMR (CDC₃) δ 11 (1H, br s, COOH), 5,07 (1H, br s, NH), 3,40 (2H, m), 2,58 (2H, m), 1,44 (9H, s, ^{t}Bu).

### Example 2

### Preparation of N-Boc-β-alanine propargyl Ester(1 b)

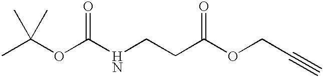

N-Boc-β-alanine (1,91 g,10.1 mmol) and propargyl alcohol (0.675 g,12 mmol) were dissolved in EtOAc (25 mL). Dicyclohexyl-carbodiimide (DCC, 2.06 g,10 mmol) was added to the solution and after 16 h of stirring at room temperature, the reaction mixture was filtered and evaporated to dryness under vacuum. Crude product yield

### Example 3

### Preparation of 5-Iodo-2′-deoxyuridine 3′,5′-Di-tert-butyldimethylsilyl Ether(1c).

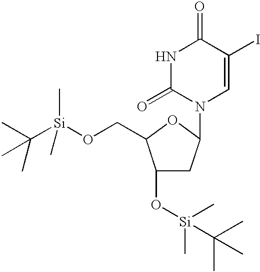

5-Iodo-2′-deoxyuridine (Aldrich, 2.39 g, 6.7 mmol) and imidazole (2.025 g, 29.7 mmol) was dissolved in anhydrous DMF (10 mL). A solution of tert-butyldimethylsilyl chloride (2.24 g, 14.9 mmol) in anhydrous DMF (5 mL) was added and the resulting mixture was stirred for 16 h at room temperature. The reaction mixture was poured into EtOAc (400 mL), washed with NH₄Cl (50% sat. aq, 80 mL) followed by water (80 mL). After drying with Na₂SO₄, EtOAc was removed under reduced pressure to leave a colourless oil that solidified on standing. Recrystallization in n-hexane (14 mL) afforded 2.64 g, 80%.

¹H NMR (CDCl₃) δ 8.18 (1H, br s, NH); 8.10 (1H, s); 6,23 (1H, dd); 4,40 (1H, dt); 4.05 (1H, dd); 3.92 (1H, dd); 3.78 (1H, dd); 2,32 (1H, ddd); 2.05 (1H, ddd); 0.95(9H, s, ^{t}Bu); 0.90(9H, s, ^{t}Bu); 0.15 (3H, s, CH₃); 0.13 (3H, s, CH₃); 0.08 (3H, s, CH₃); 0.07 (3H, s, CH₃).

### Example 4

### Preparation of Compound (1d)

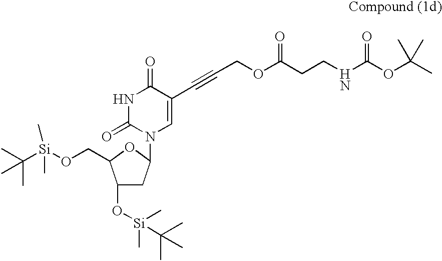

A solution of iodo silyl ether (1c) (1.62 g, 2.7 mmol), N-Boc-β-alanine(1a) (2.03 g, 8.9 mmol) and triethylamine (0.585 g, 5.8 mmol) in 10 mL dry DMF were stirred at room temperature. N₂ was passed through the solution for 20 min. Tetrakis(triphenylphosphine)palladium(0) (269 mg, 0.2 mmol) and copper(1) iodide (90 mg, 0.4 mmol) were added and the reaction mixture was stirred at room temperature for 32 h. EtOAc (100 mL) was poured into the reaction mixture, followed by washing (aq Na—HCO₃ (50 mL); brine (50 mL)), drying (Na₂SO₄), and removal of solvent by vacuum evaporation.

The crude product (2.4 g) was purified by silica column chromatography eluting with EtOAc:Heptane gradient (1:2)-(5:3) (v/v). Product yield 1.15 g, 60%.

¹H NMR (CDCl₃) δ 8.45 (1H, s), 8.05 (1H, s,6-H), 7.35 (1H, bs, NH), 6.25 (1H, dd, 1′-H), 4.82 (2H, s, CH₂O), 4,39 (1H, m, 3′-H), 3.97 (1H, m, 4′-H), 3.80 (2H, dd, 5′,5″-H), 3.40 (2H, m, CH₂N), 2.58 (2H, t, CH₂), 2,2 (1H, m, 2′-H), 2.0 (1H, m, 2″-H), 1.45 (9H, s, ^{t}Bu), 0.93 (9H, s, ^{t}Bu), 0.89 (9H, s, ^{t}Bu), 0.15 (3H, s, CH₃), 0.13 (3H, s, CH₃), 0.08 (3H, s, CH₃), 0.07 (3H, s, CH₃).

### Example 5

### Preparation of Compound (1e)

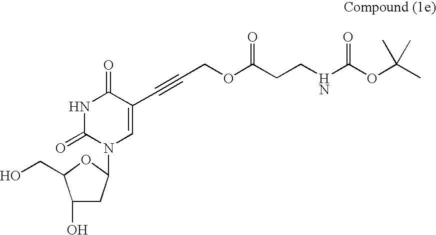

A solution of N-Boc-β-alanine silyl ether (1d) (100 mg, 0.15 mmol), glacial acetic acid (75 mg, 1.25 mmol) and tetrabutylammonium fluoride trihydrate (TBAF) (189 mg, 0.6 mmol) in 2 mL dry THF was stirred at room temperature for 3 d. The reaction mixture was evaporated and purified by silica column chromatography eluting with dichloromethane(DCM):methanol(MeOH) gradient (95:5)-(88:12) (v/v). Product yield 26 mg, 38%.

¹H NMR (CD₃OD) δ 8.35 (1H, s, 6-H), 6.15 (1H, t, 1′-H), 4.80 (2H, s, CH₂O), 4,32 (1H, dt, 3′-H), 3.86 (1H, q, 4′-H), 3.70 (2H, dd, 5′,5″-H), 3.24 (2H, m, CH₂N), 2.47 (2H, t, CH₂), 2,28-2.10 (1H, m, 2′,2″-H), 1.44 (9H, s, ^{t}Bu).

### Example 6

### Preparation of Compound (1f)

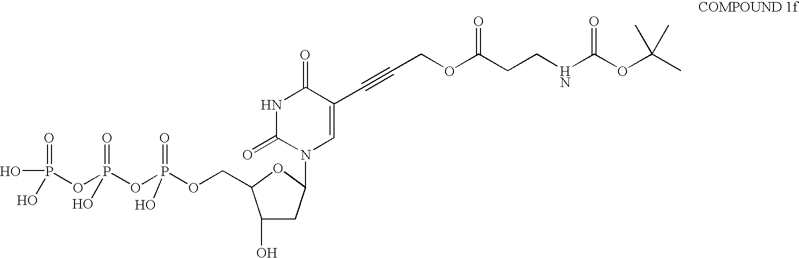

N-Boc-β-alanine nucleoside (le) (26 mg, 57 μmol) was dissolved in 200 μL dry trimethylphosphate. After cooling to 0° C., a solution of phosphorus oxychloride (POCl₃) in dry trimethylphosphate was added (100 μL stock solution (104 mg/mL), 68 μmol). The reaction mixture was stirred at 0° C. for 2 h.

Subsequently a solution of tributylammonium pyrophosphate (Sigma P-8533) (67.8 mg, 143 μmol in 300 μL dry DMF) and tributylamine (26.9 mg, 145 μmol in 150 μL dry DMF) was added at 0° C. The reaction was stirred at room temperature for 3 min. and then stopped by addition of 1 mL 1.0 M triethylammonium hydrogencarbonate.

### Example 7

### Preparation of Compound I

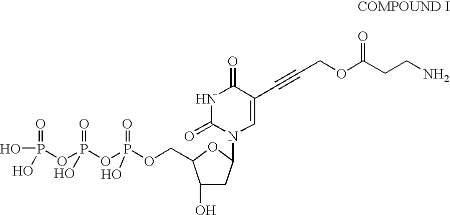

Removal of N-Boc Protection Group.

Following phosphorylation, 50 μl of the phosphorylation reaction mixture is adjusted to pH=1 using HCl and incubated at room temperature for 30 minutes. The mixture is adjusted to pH 5.5 using equimolar NaOH and Na-acetate (pH 5.5) before purification on TLC.

Purification of nucleotide derivatives using thin-layer chromatography (TLC) From the crude mixture, 20 samples of 2 μl were spotted on kieselgel 60 F₂₅₄ TLC (Merck). Organic solvents and non-phosphorylated nucleosides were separated from the nucleotides derivatives using 100% methanol as running solution. Subsequently, the TLC plate is air-dried and the nucleotide-derivative identified by UV-shadowing. Kiesel containing the nucleotide-derivative was isolated and extracted twice using 10 mM Na-acetate (pH=5.5) as solvent. Kieselgel was removed by centrifugation and the supernatant was dried in vacuo. The nucleotide derivative was resuspended in 50-100 pi H₂O to a final concentration of 1-3 mM. The concentration of each nucleotide derivative was evaluated by UV-absorption prior to use in polymerase extension reactions.

### Examples 8 to 13

### Preparation of the Mononucleotide Building Block (II)

Building block II may be prepared according to the general scheme shown below:

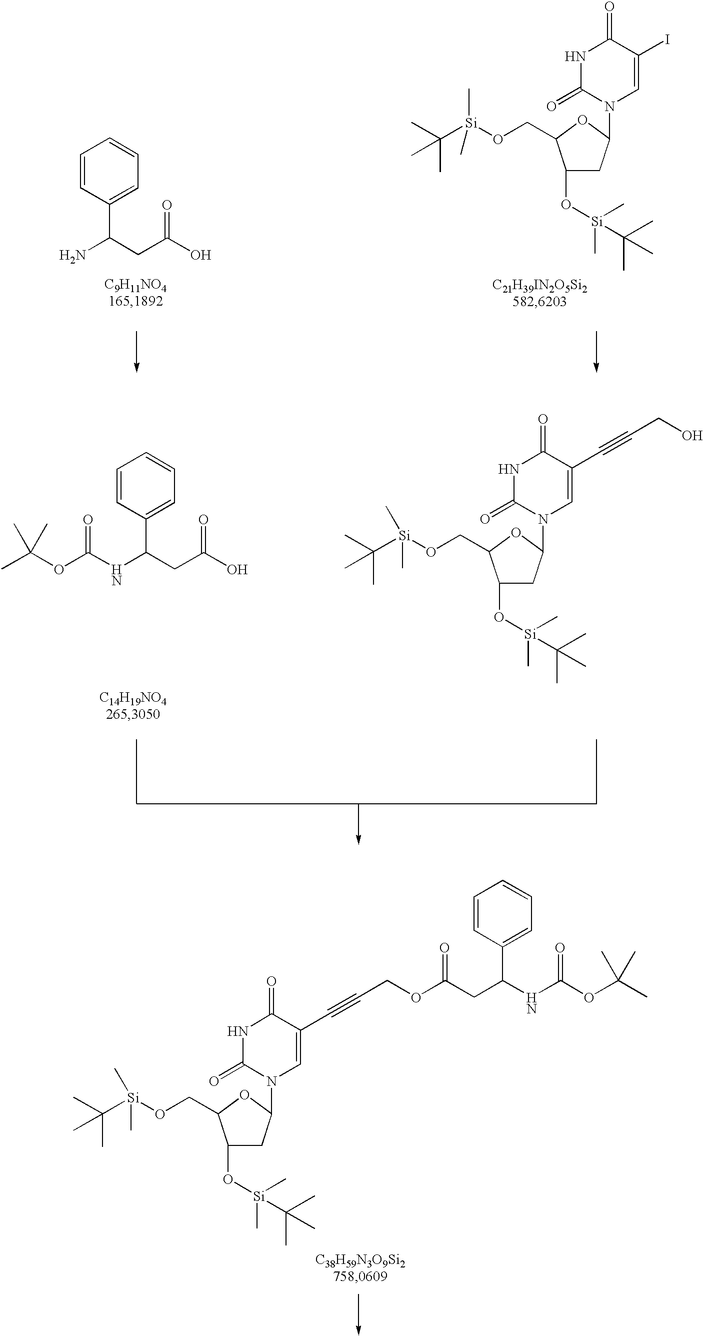

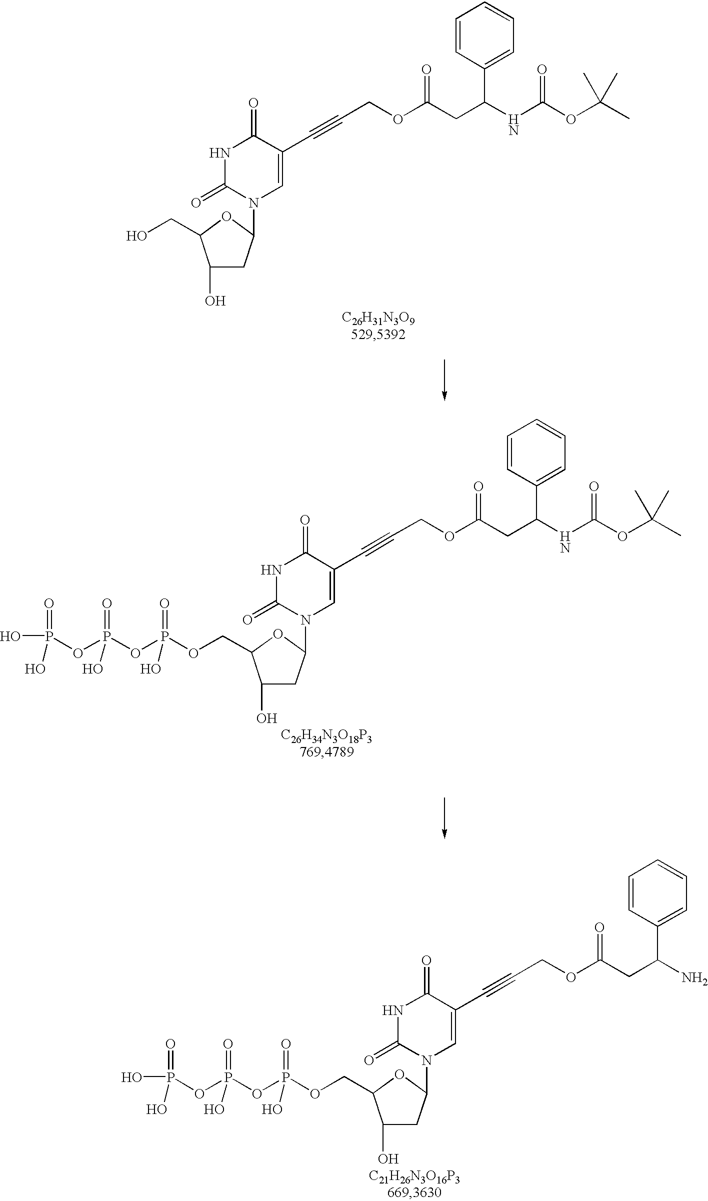

### Example 8

### Preparation of N-Boc-3-phenyl-β-alanine (2a)

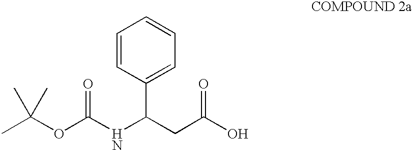

To a solution of 3-amino-3-phenylpropionic acid (3.30 g, 20 mmol) in NaHCO₃ (50% sat. aq, 25 mL) were added di-tert-butyl dicarbonate (4,36 g, 20 mmol) and acetonitrile (30 mL). The reaction mixture was stirred at room temperature for 18 h. Di-tert-butyl dicarbonate (4,36 g, 20 mmol) was added and the reaction mixture was stirred at room temperature for 18 h.

EtOAc (100 mL) was added and pH was adjusted to 4-5 by addition of NaH₂PO₄. The product was extracted into EtOAc (3×100 mL), dried (Na₂SO₄), and evaporated to dryness under vacuum to afford crude product 5.6 g (105%)

### Example 9

### Preparation of 5-(3-Hydroxypropyn-1-yl)-2′-deoxyuridine 3′,5′-Di-tert-butyldimethylsilyl Ether(2b).

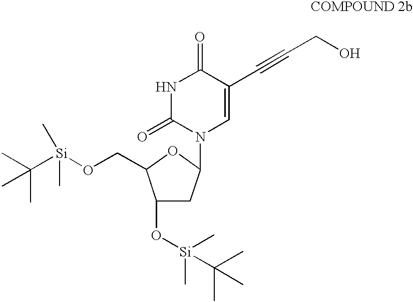

A solution of iodo silyl ether (3) (1.30 g, 2.2 mmol), propargyl alcohol (0.386 g, 6.9 mmol) and triethylamine (0.438 g, 4.3 mmol) in 7 mL dry DMF was deaeraed with N₂. Tetrakis(triphenylphosphine)palladium(0) (228 mg, 0.2 mmol) and copper(1) iodide (120 mg, 0.4 mmol) were added and the reaction mixture was stirred at room temperature for 32 h.

EtOAc (100 mL) was poured into the reaction mixture, followed by washing (aq Na—HCO₃ (50 mL); brine (50 mL)), drying (Na₂SO₄), and removal of solvent by vacuum evaporation.

The crude product (1.73 g) was purified by silica column chromatography eluting with EtOAc:Heptane gradient (2:3)-(3:2) (v/v). Product yield 0.713 g, 63%.

¹H NMR (CDCl₃) δ 8.47 (1H, s), 8.05 (1H, s, 6-H), 6.29 (1H, dd, 1′-H), 4,42 (2H, s, CH₂), 4,39 (1H, m, 3′-H), 3.98 (1H, m, 4′-H), 3.83 (2H, dd, 5′,5″-H), 2,32 (1H, m, 2′-H), 2.02 (1H, m, 2″-H), 0.93 (9H, s, ^{t}Bu), 0.89 (9H, s, ^{t}Bu), 0.15 (3H, s, CH₃), 0.13 (3H, s, CH₃), 0.08 (3H, s, CH₃), 0.07 (3H, s, CH₃)

### Example 10

### Preparation of Compound (2c)

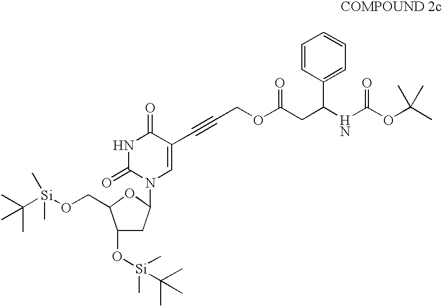

N-Boc-3-phenyl-,-alanine (8)(265 mg, 1.0 mmol) and compound (2b) (255 mg, 0.5 mmol) were dissolved in THF (15 mL). Diisopropyl-carbodiimide (DIC, 126 mg, 1 mmol) and 4-dimethylaminopyridin (DMAP, 10 mg) were added to the solution, and after 16 h of stirring at room temperature the reaction mixture was poured into EtOAc (100 mL), washed with NaHCO₃ (50% sat. aq, 50 mL), dried (Na₂SO₄), filtered and evaporated under vacuum.

The crude product was purified by silica column chromatography eluting with EtOAc:Heptane gradient (1:2)-(2:3) (v/v). Product yield 335 mg, 88%.

¹H NMR (CDCl₃) δ 8.49 (1H, s), 8.04 (1H, s, 6-H), 7.29 (5H, m, Ph), 6.27 (1H, dd, 1′-H), 5.5 (1H, bd), 5.09 (1H,m), 4,80 (2H, s, CH₂), 4,39 (1H, m, 3′-H), 3.98 (1H, m, 4′-H), 3.82 (2H, dd, 5′,5″-H), 2,87 (2H, d), 2.29 (1H, m, 2′-H), 2.01 (1H, m, 2″-H), 1.41 (9H, s, ^{t}Bu), 0.91 (9H, s, ^{t}Bu), 0.89 (9H, s, ^{t}Bu), 0.15 (3H, s, CH₃), 0.13 (3H, s, CH₃), 0.08 (3H, s, CH₃), 0.07 (3H, s, CH₃).

### Example 11

### Preparation of Compound 2d

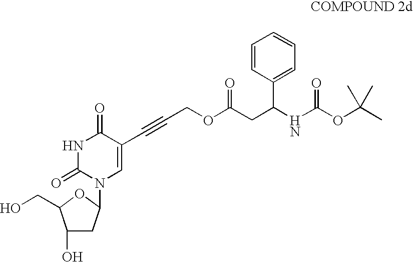

A solution of compound (2c) (334 mg, 440 μmol), glacial acetic acid (190 mg, 3.15 mmol) and tetrabutylammonium fluoride trihydrate (TBAF) (500 mg, 1.58 mmol) in 6 mL dry THF was stirred at room temperature for 18 h.

The reaction mixture was evaporated and purified by silica column chromatography eluting with (DCM):(MeOH) gradient (95:5)-(9:1) (v/v). Product yield 122 mg, 52%.

¹H NMR (CDCl₃) δ 10.1 (1H, s), 8.24 (1H, s, 6-H), 7.3 (5H, m, Ph), 6.37 (1H, dd, 1′-H), 5.6 (1H, bs), 5.09 (1H,m), 4,79 (2H, s, CH₂), 4,52 (1H, m, 3′-H), 4.0 (1H, m, 4′-H), 3.85 (2H, dd, 5′,5″-H), 2,87 (2H, d), 2.4 (1H, m, 2′-H), 2.25 (1H, m, 2″-H), 1.4 (9H, s, ^{t}Bu).

### Example 12

### Preparation of Compound (2e)

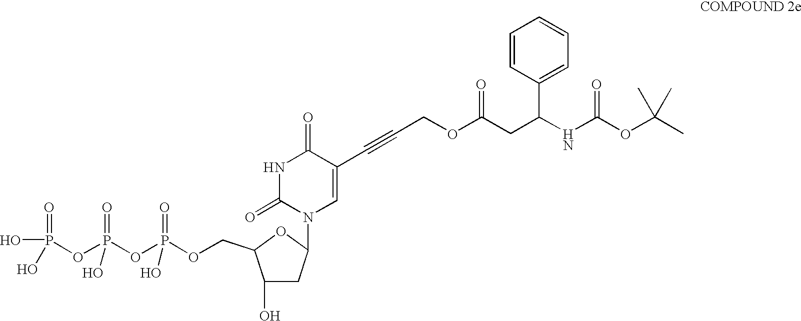

Compound (2d) (122 mg, 230 Fmol) was dissolved in 400 AL dry trimethylphosphate. After cooling to 0° C., a solution of phosphorus oxychloride (POCl₃) in dry trimethylphosphate was added (400 μL stock solution (105 mg/mL), 276 μmol). The reaction mixture was stirred at 0° C. for 2 h. Subsequently a solution of tributylammonium pyrophosphate (273 mg, 576 μmol in 1.2 mL dry DMF) and tributylamine (109 mg, 587 μmol in 600 μL dry DMF) was added at 0° C. The reaction was stirred at room temperature for 10 min. and then stopped by addition of 1.0 M triethylammonium hydrogencarbonate (1 mL).

### Example 13

### Preparation of Compound II

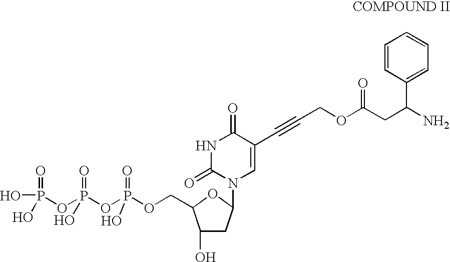

Removal of N-Boc Protection Group.

Following phosphorylation, 50 μl of the phosphorylation reaction mixture is adjusted to pH=1 using HCl and incubated at room temperature for 30 minutes. The mixture is adjusted to pH 5.5 using equimolar NaOH and Na-acetate (pH 5.5) before purification on TLC.

Purification of Nucleotide Derivatives Using Thin-Layer Chromatography (TLC)

From the crude mixture, 20 samples of 2 pl were spotted on kieselgel 60 F₂₅₄TLC (Merck). Organic solvents and non-phosphorylated nucleosides were separated from the nucleotides derivatives using 100% methanol as running solution. Subsequently, the TLC plate is air-dried and the nucleotide-derivative identified by UV-shadowing. Kiesel containing the nucleotide-derivative was isolated and extracted twice using 10 mM Na-acetate (pH=5.5) as solvent. Kieselgel was removed by centrifugation and the supernatant was dried in vacuo. The nucleotide derivative was resuspended in 50-100 μl H₂O to a final concentration of 1-3 mM. The concentration of each nucleotide derivative was evaluated by UV-absorption prior to use in polymerase extension reactions.

### Examples 14 to 18

### Preparation of the Mononucleotide Building Block (III)

Building block III may be prepared according to the general scheme shown below:

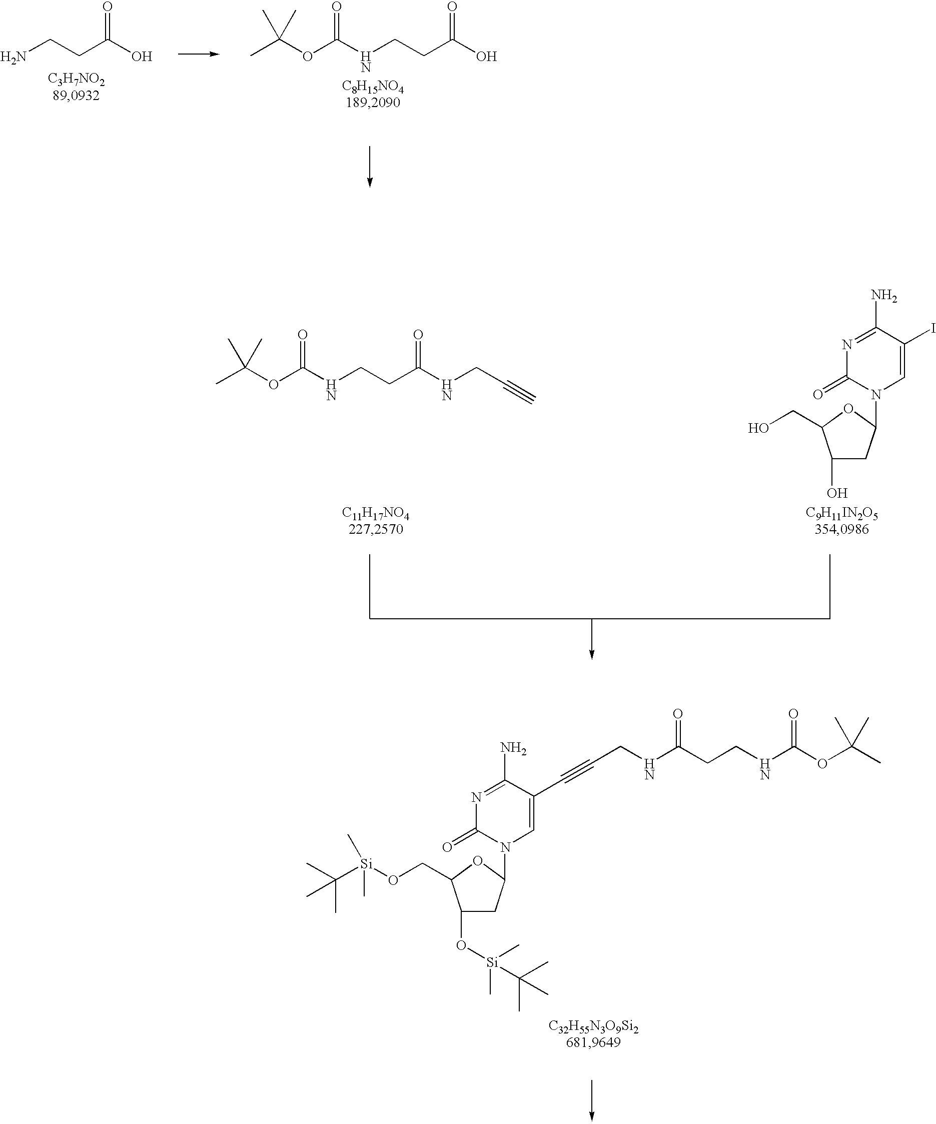

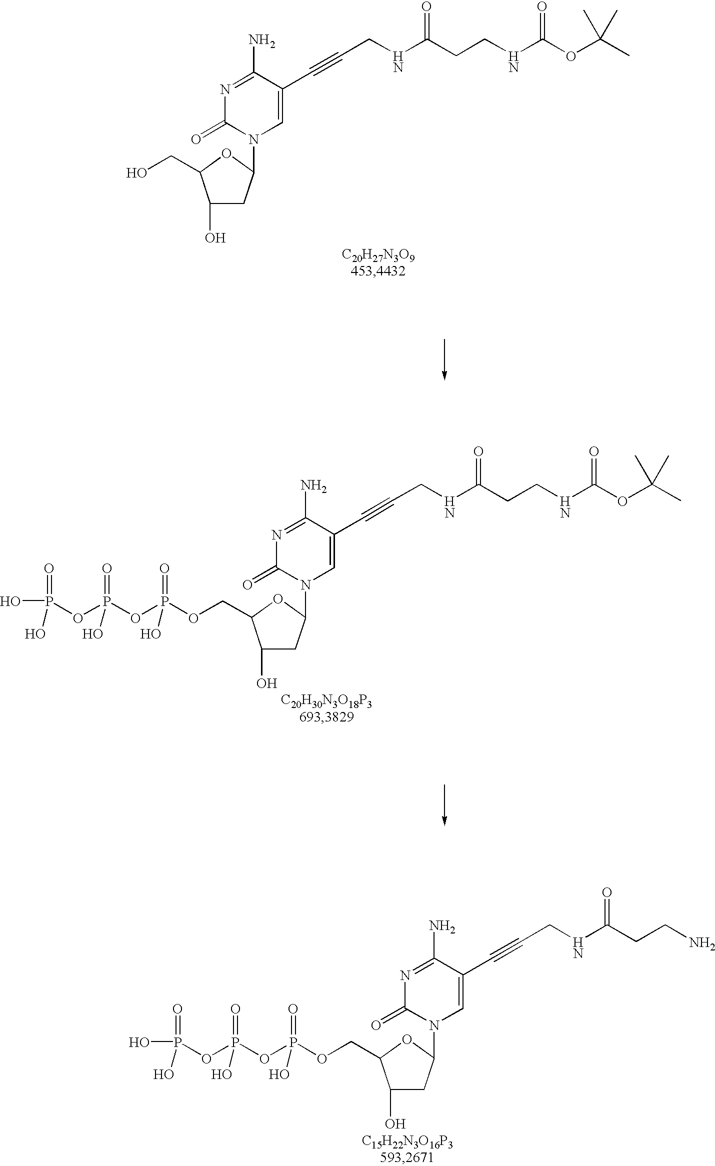

### Example 14

### Preparation of N-Boc-β-Alanine Propargyl Amide(3a)

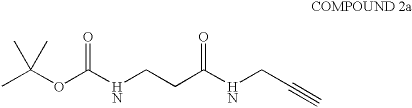

N-Boc-β-alanine(1a) (1,05 g, 5.5 mmol) and propargyl amine (0.90 g, 16.5 mmol) were dissolved in THF (10 mL). Diisopropyl-carbodiimide (DIC, 695 g, 5.5 mmol) was added and the reaction mixture was stirred for 16 h at room temperature. Water was added (20 mL) and the product was extracted into EtOAc (3×30 mL). The combined EtOAc was dried (Na₂SO₄) and evaporated. The crude product was purified by silica column chromatography eluting with EtOAc:Heptane gradient (2:3)-(3:2.5) (v/v). Product yield 0.925 g, 74%.

¹H NMR (CDCl₃) δ 6.69 (1H, bs, NH), 5,32 (1H, bs, NH), 4.04 (2H, bs), 3,41 (2H, dd), 2,45 (2H, t), 2.24 (1H, s), 1,44 (9H, s, ^{t}Bu).

### Example 15

### Preparation of Compound (3b)

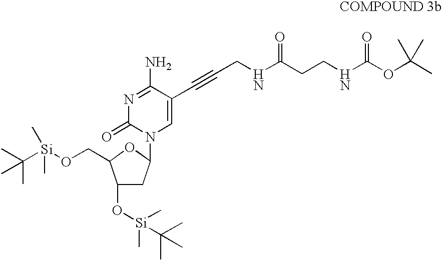

A solution of 5-iodo-2′-deoxycytidine (176 mg, 0.5 mmol), N-Boc-,-alanine propargyl amide(14) and triethylamine (100 mg, 1.0 mmol) in dry DMF (5 mL) were stirred at room temperature. N₂ was passed through the solution for 20 min. Tetrakis(triphenylphosphine)palladium(0) (66.5 mg, 0.057 mmol) and copper(1) iodide (20.7 mg, 0.108 mmol) were added and the reaction mixture was stirred at room temperature for 5 d Imidazole (112 mg, 1.6 mmol)was added. A solution of tert-butyldimethylsilyl chloride (234 mg, 1.5 mmol) in anhydrous DMF (1 mL) was added and the resulting mixture was stirred for 16 h at room temperature.

The reaction mixture was evaporated and EtOAc (25 mL) was added. The resulting mixture was filtrated and the solvent removed by vacuum evaporation. The crude product was purified by silica column chromatography eluting with DCM:MeOH (92.5-7.5) (v/v). Product yield 84 mg, 25%.

¹H NMR (CDCl₃) δ 8.13 (H, s), 6.21 (1H, dd, 1′-H), 4.66 (1H, m), 4,16 (2H, s, CH₂), 4,04-3.85 (4H, m), 3.35-3.31 (2H, m), 2,43-2.36 (2H, m), 2.12-1.99 (1H, m), 1.44 (9H, s, ^{t}Bu), 0.95 (9H, s, ^{t}Bu), 0.92 (9H, s, ^{t}Bu), 0.17 (3H, s, CH₃), 0.15 (3H, s, CH₃), 0.13 (3H, s, CH₃), 0.12 (3H, s, CH₃).

### Example 16

### Preparation of Compound (3c)

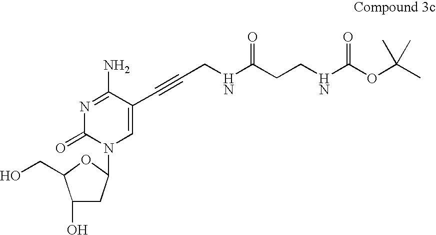

A solution of compound(3b) (84 mg, 0.12 mmol) and tetrabutylammonium fluoride trihydrate (TBAF) (155 mg, 0.45 mmol) in 2 mL dry THF was stirred at room temperature for 4 days.

The reaction mixture was evaporated and purified by silica column chromatography eluting with DCM:MeOH gradient (9:1)-(8:2) (v/v). Product yield 27 mg, 48%.

¹H NMR (CDCl₃) δ 8.32 (1H, s), 6.20 (1H, dd, 1′-H), 4.35 (1H, dt), 4,15 (2H, s, CH₂), 3.95 (1H, q), 3.83 (1H, dd), 3.72 (1H, dd), 3,36-3.30 (3H, m), 2.42-2.36 (3H, m), 2.13 (1H, dt), 1.40 (9H, s, ^{t}Bu).

### Example 17

### Preparation of Compound (3d)

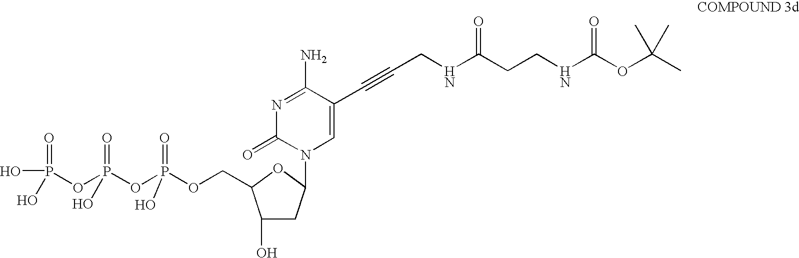

Compound (3c) (27 mg, 60 μmol) was dissolved in 100 IL dry trimethylphosphate. After cooling to 0° C., a solution of phosphorus oxychloride (POCl₃) in dry trimethylphosphate was added (100 μL stock solution (110 mg/mL), 72 μmol). The reaction mixture was stirred at 0° C. for 2 h.

Subsequently a solution of tributylammonium pyrophosphate (71 mg, 150 μmol in 300 μL dry DMF) and tributylamine (28.3 mg, 153 μmol in 150 μL dry DMF) was added at 0° C. The reaction was stirred at room temperature for 3 min. and then stopped by addition of 1.0 M triethylammonium hydrogencarbonate (1 mL).

### Example 18

### Preparation of Compound III

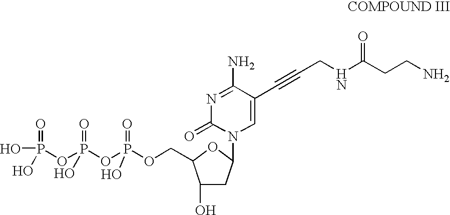

Removal of N-Boc Protection Group.

Following phosphorylation, 50 μl of the phosphorylation reaction mixture is adjusted to pH=1 using HCl and incubated at room temperature for 30 minutes. The mixture is adjusted to pH 5.5 using equimolar NaOH and Na-acetate (pH 5.5) before purification on TLC.

Purification of Nucleotide Derivatives using Thin-Layer Chromatography (TLC)

From the crude mixture, 20 samples of 2 μl were spotted on kieselgel 60 F₂₅₄ TLC (Merck). Organic solvents and non-phosphorylated nucleosides were separated from the nucleotides derivatives using 100% methanol as running solution. Subsequently, the TLC plate is air-dried and the nucleotide-derivative identified by UV-shadowing. Kiesel containing the nucleotide-derivative was isolated and extracted twice using 10 mM Na-acetate (pH=5.5) as solvent. Kieselgel was removed by centrifugation and the supernatant was dried in vacuo. The nucleotide derivative was resuspended in 50-100 pi H₂O to a final concentration of 1-3 mM. The concentration of each nucleotide derivative was evaluated by UV-absorption prior to use in polymerase extension reactions.

### Examples 19 to 22

### Preparation of the Mononucleotide Building Block (IV)

Building block IV may be prepared according to the general scheme shown below:

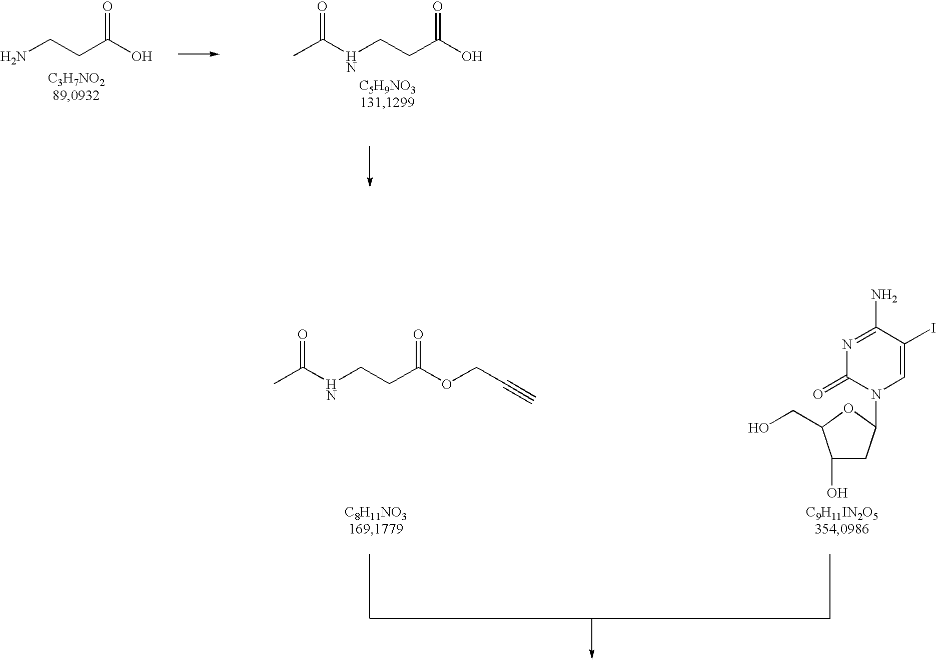

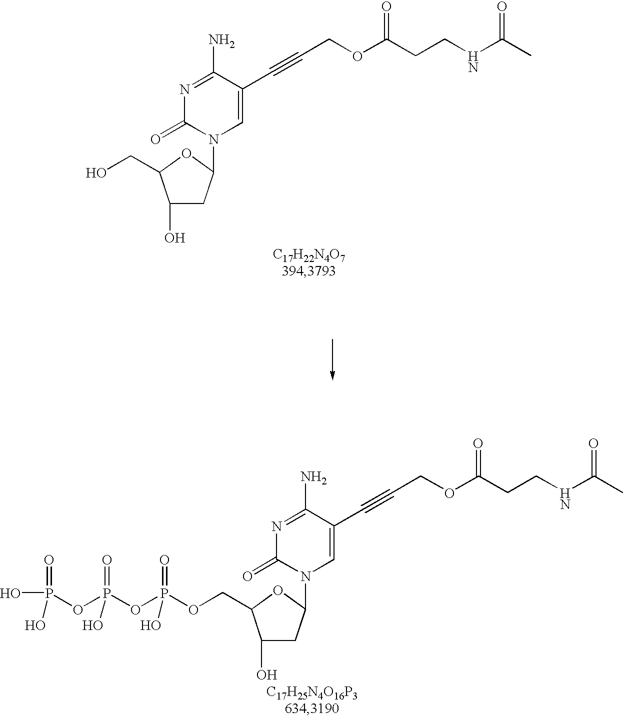

### Example 19

### Preparation of N-Acetyl-β-alanine(4a)

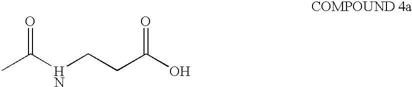

To a solution of β-alanine (2,25 g, 25 mmol) in aq. NaHCO₃(15 mL) was added acetonitrile (15 mL) and acetic anhydride (2.55 g, 25 mmol). The reaction mixture was stirred at room temperature for 3 h. Acetic anhydride (2.55 g, 25 mmol) was added and after 2 h and pH was adjusted to 4-5 by addition of NaH₂PO₄.

The product was extracted into EtOAc (3×50 mL), dried (Na₂SO₄), and evaporated to dryness under vacuum to afford 1.96 g (60%)

### Example 20

### Preparation of N-Acetyl-β-alanine Propargyl Ester(4b)

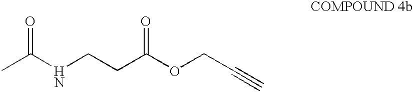

To a solution of N-Acetyl-β-alanine(4a) in THF (20 mL) was added propargyl alcohol (840 mg, 15 mmol), 1-(3-dimethylaminopropyl)-3-ethylcarbodiimide hydrochloride (EDC) (1.035 g,5.39 mmol), triethylamine (540 mg, 5.4 mmol) and 4-dimethylaminopyridin (5 mg). The reaction mixture was stirred at room temperature for 2 d.

The reaction mixture was poured into EtOAc (100 mL), washed with NaH₂PO₄ (50% sat. aq, 2×50 mL) followed by NaHCO₃ (50% sat. aq, 50 mL). After drying (Na₂SO₄), EtOAc was removed under reduced pressure to leave a colourless oil that solidified on standing. Product yield 536 mg, 59%.

### Example 21

### Preparation of Compound (4c)

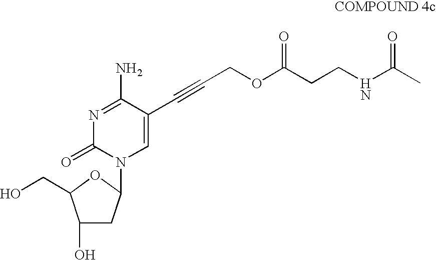

A solution of 5-iodo-2′-deoxycytidin (200 mg, 0.56 mmol), triethylamine (100 mg, 1 mmol) and compound (4b) (190 mg, 1.13 mmol) in anhydrous DMF (7 mL) was stirred at room temperature. N₂ was passed through the solution for 20 min. Tetrakis(triphenylphosphine)palladium(0) (70 mg, 0.06 mmol) and copper(1) iodide (22 mg, 0.12 mmol) were added and the reaction mixture was stirred at room temperature for 4 d.

The reaction mixture was evaporated and purified by silica column chromatography eluting with DCM:MeOH gradient (9:1)-(8:2) (v/v). Product yield 141 mg, 63%.

¹H NMR (CD₃OD) δ 8.41 (1H, s), 6.20 (1H, dd, 1′-H), 4.97 (2H, s), 4.38 (1H, dt), 3.97 (1H, q), 3.85 (1H, dd), 3.75 (1H, dd), 3,46 (2H, t), 2.61 (2H, t), 2.39 (1H, m), 2.18 (1H, m).

### Example 22

### Preparation of Compound IV

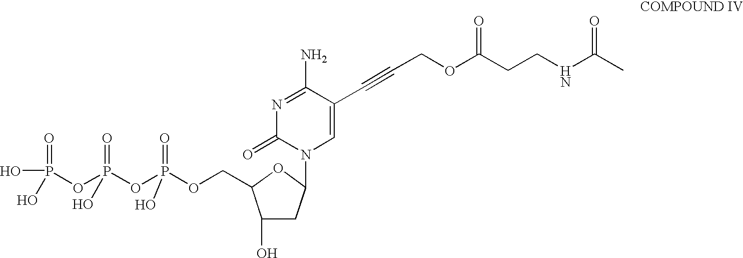

Compound (4c) (140 mg, 355 μmol) was dissolved in 600 μL dry trimethylphosphate. After cooling to 0° C., a solution of phosphorus oxychloride (POCl₃) in dry trimethylphosphate was added (600 μL stock solution (108 mg/mL), 420 μmol). The reaction mixture was stirred at 0° C. for 2 h.

Subsequently a solution of tributylammonium pyrophosphate (422 mg, 890 μmol in 1.8 mL dry DMF) and tributylamine (168 mg, 900 μmol in 0.9 mL dry DMF) was added at 0° C. The reaction was stirred at room temperature for 3 min. and then stopped by addition of 1.0 M triethylammonium hydrogencarbonate (1 mL).

From the crude mixture, 20 samples of 2 μl were spotted on kieselgel 60 F₂₅₄TLC (Merck). Organic solvents and non-phosphorylated nucleosides were separated from the nucleotides derivatives using 100% methanol as running solution. Subsequently, the TLC plate is air-dried and the nucleotide-derivative identified by UV-shadowing. Kiesel containing the nucleotide-derivative was isolated and extracted twice using 10 mM Na-acetate (pH=5.5) as solvent. Kieselgel was removed by centrifugation and the supernatant was dried in vacuo. The nucleotide derivative was resuspended in 50-100 μl H₂O to a final concentration of 1-3 mM. The concentration of each nucleotide derivative was evaluated by UV-absorption prior to use in polymerase extension reactions.

### Examples 23 to 28

### Preparation of the Mononucleotide Building Block (V)

Building block V may be prepared according to the general scheme shown below:

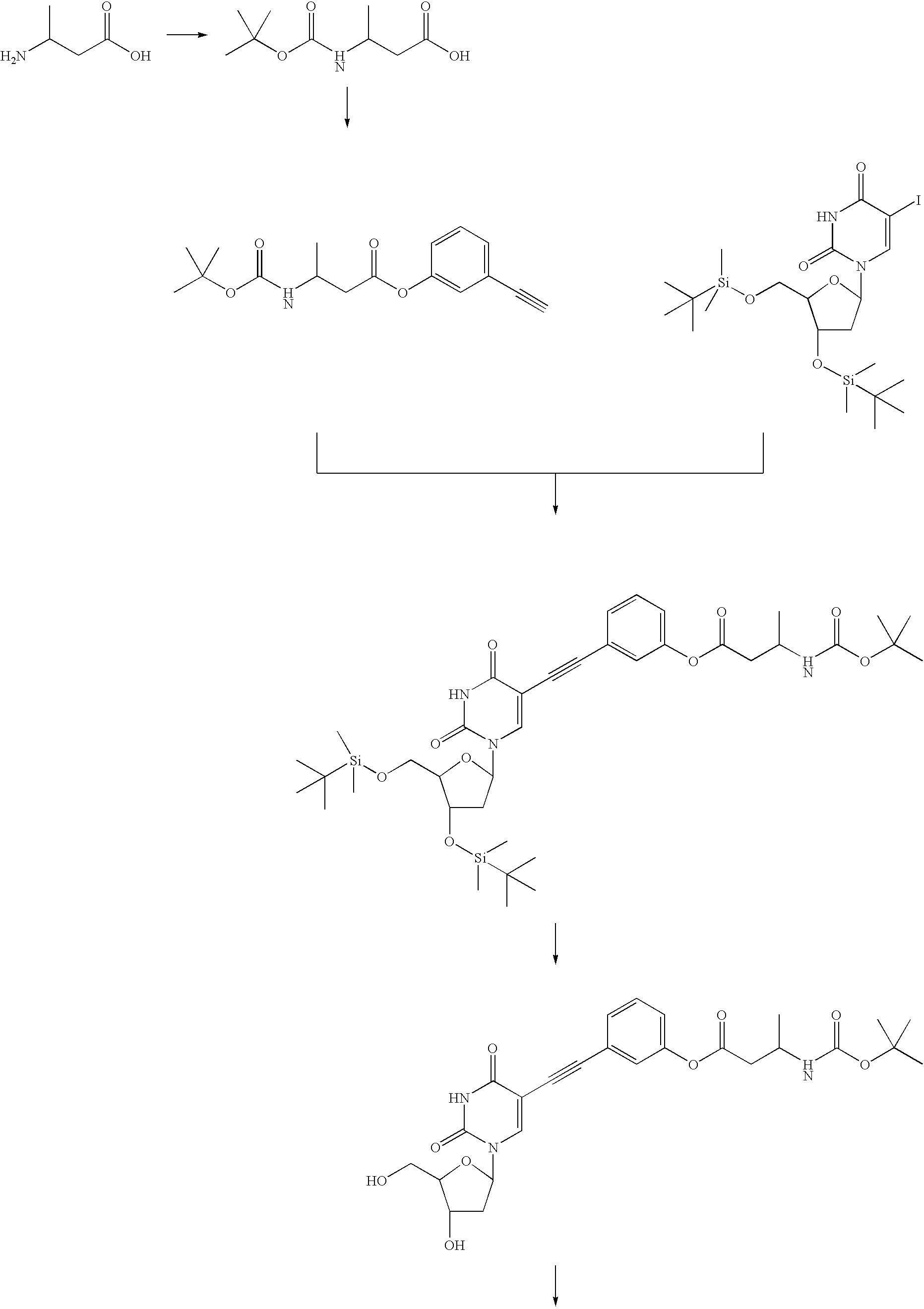

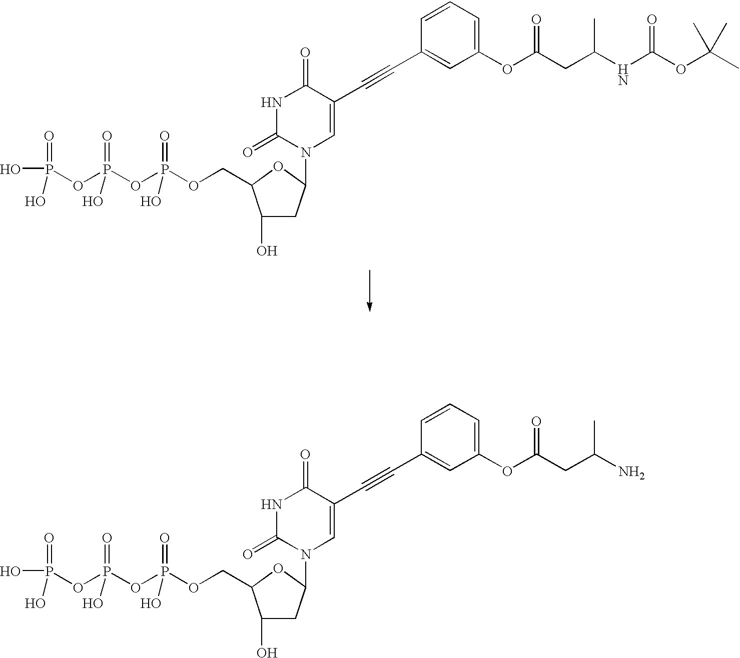

### Example 23

### Preparation of compound 5a

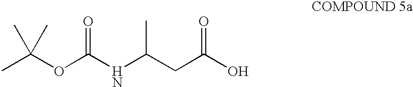

To a solution of 3-amino-butyric acid (2.06 g, 20 mmol) in NaHCO₃ (50% sat. aq, 25 mL) were added di-tert-butyl dicarbonate (4,36 g, 20 mmol) and acetonitrile (30 mL). The reaction mixture was stirred at room temperature for 18 h. Di-tert-butyl dicarbonate (4,36 g, 20 mmol) was added and the reaction mixture was stirred at room temperature for 18 h.

EtOAc (100 mL) was added and pH was adjusted to 4-5 by addition of NaH₂PO₄. The product was extracted into EtOAc (3×100 mL), dried (Na₂SO₄), and evaporated to dryness under vacuum to afford crude product 4.6 g (113%).

### Example 24

### Preparation of Compound 5b

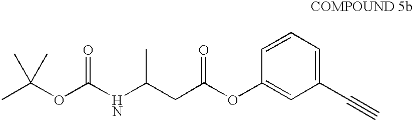

Compound 28 (1,023 g, 5.0 mmol), 3-Ethynyl-phenole (Lancaster, 0.675 g, 12 mmol) and 4-dimethylamino-pyridin (DMAP, 300 mg, 2.5 mmol) were dissolved in EtOAc (10 mL). Dicyclohexyl-carbodiimide (DCC, 2.06 g, 10 mmol) was added to the solution and after 16 h of stirring at room temperature, the reaction mixture was filtered and evaporated to dryness under vacuum. The crude product was purified by silica column chromatography eluting with EtOAc:Heptane gradient (1:3)-(1:2)(v/v). Product yield 720 mg, 73%.

¹H NMR (CDCl₃) δ 7.36-7.09 (4H, m, Ph), 4.89 (1H, bs, NH), 4.22 (1H, bm,CH), 3.10 (1H, s), 2.77 (2H, d), 1.40 (3H, t), 1.32 (3H, d).

### Example 25

### Preparation of Compound 5c

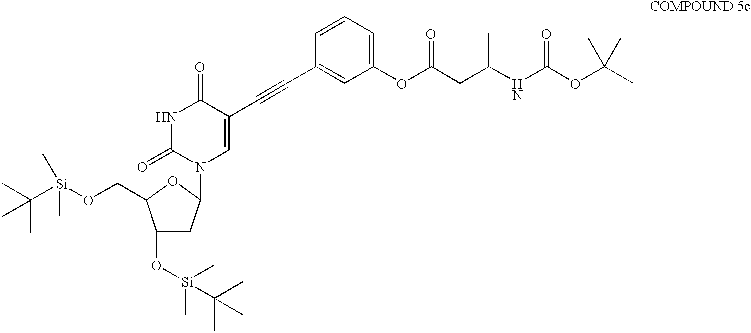

A solution of 5-Iodo-2′-deoxyuridine 3′,5′-Di-tert-butyldimethylsilyl ether (730 mg, 1.25 mmol), triethylamine (250 mg, 2.5 mmol) and compound(5b) (456 mg, 1.5 mmol) in anhydrous DMF (3 mL) was stirred at room temperature. N₂ was passed through the solution for 20 min. Tetrakis(triphenylphosphine)palladium(0) (109 mg, 0.094 mmol) and copper(1) iodide (36 mg, 0.188 mmol) were added and the reaction mixture was stirred at room temperature for 3 d.

The reaction mixture was evaporated and purified by silica column chromatography eluting with EtOAc:Heptane gradient (1:3)-(1:2)(v/v). Product yield 807 mg, 85%.

¹H NMR (CDCl₃) δ 8.38 (1H, s), 8.08 (1H, s, 6-H), 7.39-7.1 (4H, m, Ph), 6.33 (1H, dd, 1′-H), 4.9 (1H, bs), 4.45 (1H, dt), 4,80 (2H, s, CH₂), 4,2 (1H, m), 4.02 (1H, m, 4′-H), 3.95 (1H, dd, 5′-H), 3.79 (1H, dd, 5″-H), 2,78 (2H, d), 2.36 (1H, m, 2′-H), 2.07 (1H, m, 2″-H), 1.46 (9H, s, ^{t}Bu), 0.93 (9H, s, ^{t}Bu), 0.91 (9H, s, ^{t}Bu), 0.15 (3H, s, CH₃), 0.13 (3H, s, CH₃), 0.11 (3H, s, CH₃), 0.09 (3H, s, CH₃).

### Example 26

### Preparation of Compound 5d

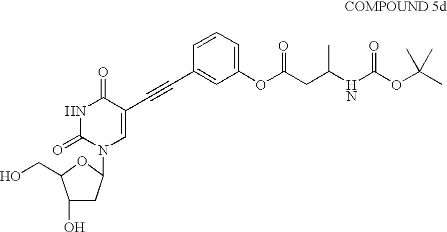

A solution of compound (5c) (807 mg, 1.06 mmol), glacial acetic acid (1.0 g, 16 mmol) and tetrabutylammonium fluoride trihydrate (TBAF) (2.36 g, 7.5 mmol) in 20 mL dry THF was stirred at room temperature for 3 d.

The reaction mixture was evaporated and purified by silica column chromatography eluting with (DCM):(MeOH) (9:1) (v/v). Product yield 408 mg, 72%.

¹H NMR (CD₃OD) δ 8.46 (1H, s, 6-H), 7.39 (2H, m, Ph), 7.28 (1H, m, Ph), 7.12 (1H, m, Ph), 6.75 (1H, bd), 6.27 (1H, dd, 1′-H), 4.44 (1H, dt, 4′-H), 3.96 (1H, t, 3′-H), 3.86 (1H, dd, 5′-H), 3.77 (1H, dd, 5″-H), 2,72 (2H, d), 2.35-2.27 (2H, m, 2′, 2″-H), 1.46 (9H, s, ^{t}Bu), 1.27 (3H, d).

### Example 27

### Preparation of Compound 5e

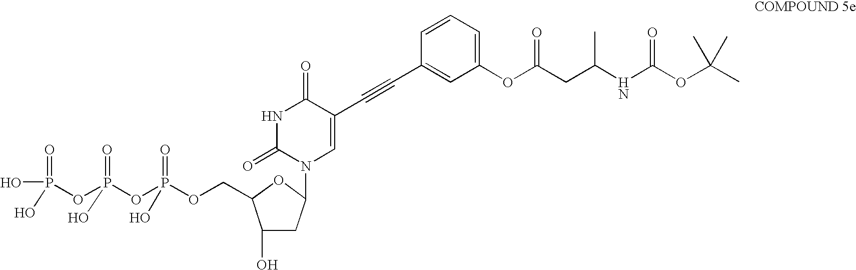

Compound (5d) (138.5 mg, 260,mol) was dissolved in 500 μL dry trimethylphosphate. After cooling to 0° C., a solution of phosphorus oxychloride (POCl₃) in dry trimethylphosphate was added (400 μL stock solution (120 mg/mL), 310 μmol). The reaction mixture was stirred at 0° C. for 2 h.

Subsequently a solution of tributylammoniumpyrophosphate (200 mg, 420 μmol in 1.00 mL dry DMF) and tributylamine (123 mg, 670 μmol in 500 μL dry DMF) was added at 0° C. The reaction was stirred at room temperature for 3 min. and then stopped by addition of 1 mL 1.0 M triethylammoniumhydrogencarbonate.

### Example 28

### Preparation of Compound V

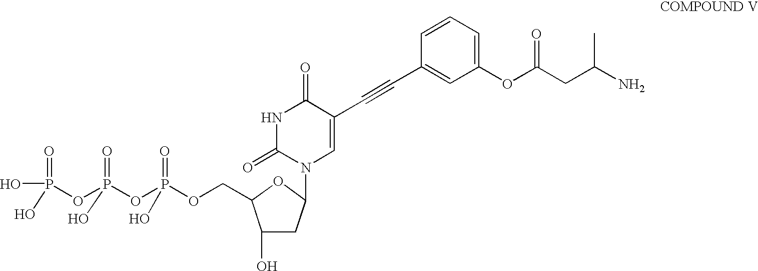

Removal of N-Boc Protection Group.

Following phosphorylation, 50 μl of the phosphorylation reaction mixture is adjusted to pH=1 using HCl and incubated at room temperature for 30 minutes. The mixture is adjusted to pH 5.5 using equimolar NaOH and Na-acetate (pH 5.5) before purification on TLC.

Purification of Nucleotide Derivatives using Thin-Layer Chromatography (TLC)

From the crude mixture, 20 samples of 2 μl were spotted on kieselgel 60 F₂₅₄ TLC (Merck). Organic solvents and non-phosphorylated nucleosides were separated from the nucleotides derivatives using 100% methanol as running solution. Subsequently, the TLC plate is air-dried and the nucleotide-derivative identified by UV-shadowing. Kiesel containing the nucleotide-derivative was isolated and extracted twice using 10 mM Na-acetate (pH=5.5) as solvent. Kieselgel was removed by centrifugation and the supernatant was dried in vacuo. The nucleotide derivative was resuspended in 50-100 μl H₂O to a final concentration of 1-3 mM. The concentration of each nucleotide derivative was evaluated by UV-absorption prior to use in polymerase extension reactions.

### Examples 29 to 31

### Preparation of the Mononucleotide Building Block (VI)

### Example 29

### Preparation of Pent-4-ynoic acid 4-oxo-4H-benzo[d][1,2,3]triazin-3-yl ester (6a)

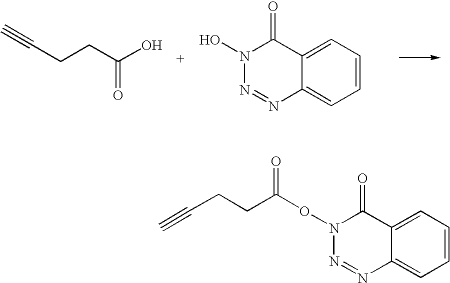

Pentynoic acid (200 mg, 2.04 mmol) was dissolved in THF (4 mL). The solution was cooled in a brine-icewater bath. A solution of dicyclohexylcarbodiimide (421 mg, 2.04 mmol) in THF (2 mL) was added. 3-Hydroxy-1,2,3-benzotriazin-4(3H)-one (333 mg, 2.04 mmol) was added after 5 minutes. The reaction mixture was stirred 1 h at −10° C. and then 2 h at room temperature. TLC indicated full conversion of 3-hydroxy-1,2,3-benzotriazin-4(3H)-one (eluent: ethyl acetate). Precipitated salts were filtered off. The filtrate was concentrated in vacuo and crystallized from hexane (4 mL). The crystals were filtered off and dried. Yield: 450 mg, 93%. R_{F}=0.8 (ethyl acetate).

### Example 30

### Preparation of 2-Pent4-ynoylamino-succinic Acid 1-tert-butyl ester 4-isopropyl Ester (6b)

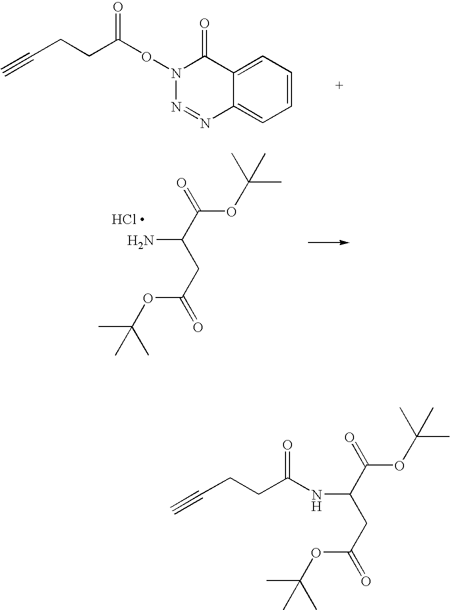

L-Aspartic acid α,β-di-tert-butyl ester hydrochloride (Novabiochem 04-12-5066, 200 mg, 0.71 mmol) was dissolved in THF (5 mL). The activated ester 6a (173 mg, 0.71 mmol) and diisopropylethylamine (0.15 mL, 0.86 mmol) were added. The mixture was stirred overnight. Dichloromethane (10 mL) was added. The organic phase was washed with citric acid (2×10 mL), saturated NaHCO₃ (aq, 10 mL), brine (10 mL), dried (Na₂SO₄) and concentrated to a syrup. An NMR spectrum indicated the syrup was pure enough for further synthesis. ¹H-NMR (CDCl₃): δ 6.6 (1H, NH), 4.6 (1H, CH), 2.8 (2H, CH₂), 2.4 (4H, 2×CH₂), 1.9 (1H, CH), 1.2 (18H, 6×CH₃).

### Example 31

### Preparation of 2-{5-[1-(4-Hydroxy-5-(O-triphosphate-hydroxymethyl)-tetrahydrofuran-2-yl)-2,4-dioxo-1,2,3,4-tetrahydro-pyrimidin-5-yl]-pent-4-ynoylamino}-succinic acid di-tert-butyl Ester (VI)

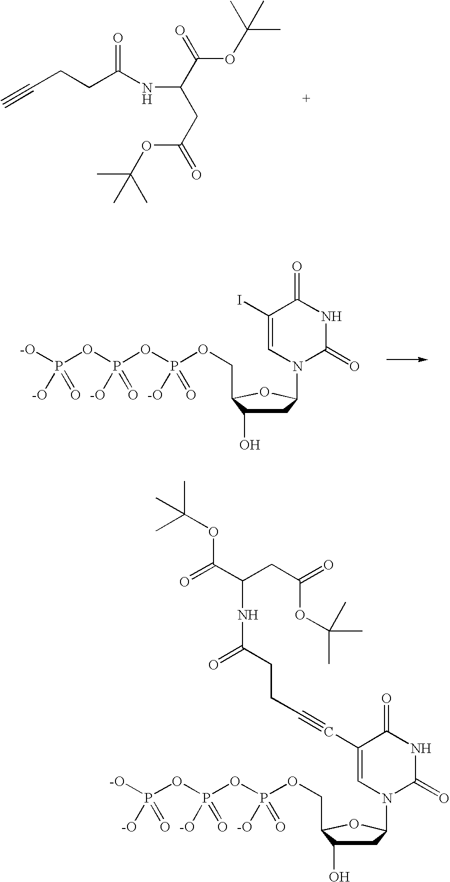

The nucleotide 9d (20 mg, 0.022 mmol) was dissolved in water-ethanol (1:1, 2 mL). The solution was degassed and kept under an atmosphere of argon. The catalyst Pd(PPh₂(m-C₆H₅SO₃Na⁺))₄ (20 mg, 0.016 mmol) prepared in accordance with A. L. Casalnuovo et al. J. Am. Chem. Soc. 1990, 112, 4324-4330, triethylamine (0.02 mL, 0.1 mmol) and the alkyne 6b (20 mg, 0.061 mmol) were added. Few crystals of CuI were added. The reaction mixture was stirred for 6 h. The triethylammonium salt of compound VI was achieved after purification by RP-HPLC (eluent: 100 mM triethylammonium acetate→20% acetonitrile in 100 mM triethylammonium acetate). ¹H-NMR (D₂O): δ 8.1 (1H, CH), 6.2 (1H, CH), 4.8 (1H, CH), 4.6 (1H, CH), 4.1 (3H, CH, CH₂), 2.8 (2H, CH₂), 2.7 (2H, CH₂), 2.5 (2H, CH₂), 2.3 (2H, CH₂), 1.4 (18H, 6×CH₃).

Immediately prior to incorporation or after incorporation, the protective di-tert-butyl ester groups may be cleaved to obtain the corresponding free carboxylic acid.

### Examples 32 to 33

### Preparation of the Mononucleotide Building Block (VII)

### Example 32

### Preparation of 2-{5-[4-Amino-1-(4-hydroxy-5-hydroxymethyl-tetrahydrofuran-2-yl)-2-oxo-1,2-dihydro-pyrimidin-5-yl]-pent-4-ynoylamino}-succinic Acid di-tert-butyl Ester (7a)

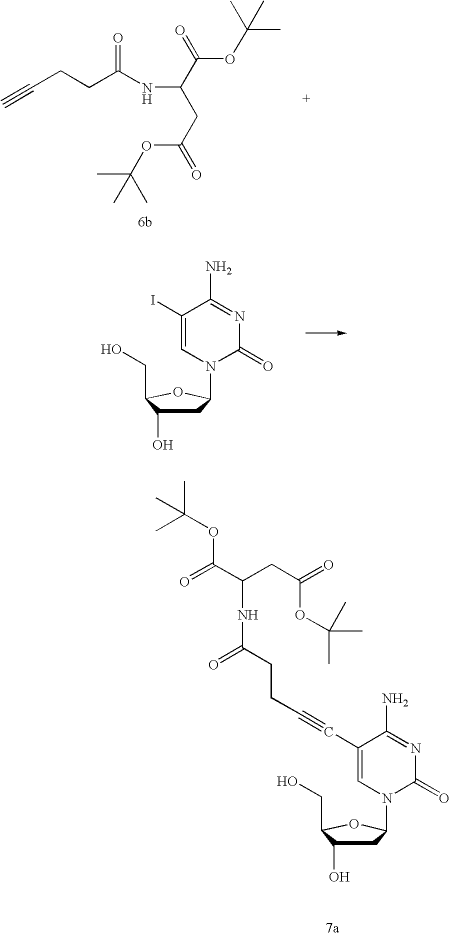

Compound (7a) (30 mg, 19%) was obtained from compound (6b) (140 mg, 0.43 mmol) and 5-iodo-2-deoxycytidine (100 mg, 0.28 mmol) using the procedure described for the synthesis of compound VI. ¹H-NMR (MeOD-D₃): δ 8.3 (1H, CH), 6.2 (1H, CH), 4.8 (1H, CH), 4.6 (1H, CH), 4.4 (1H, CH), 4.0 (1H, CH), 3.8 (2H, CH₂), 2.8 (4H, 2×CH₂), 2.7 (2H, CH₂), 2.4 (1H, CH₂), 2.2 (1H, CH₂), 1.4 (18H, 6×CH₃).

### Example 32

### Preparation of 2-{5-[4-Amino-1-(4-hydroxy-5-(O-triphosphate-hydroxymethyl)-tetrahydro-furan-2-yl)-2-oxo-1,2-dihydro-pyrimidin-5-yl]-pent-4-ynoylamino}-succinic Acid di-tert-butyl Ester (Compound VII)

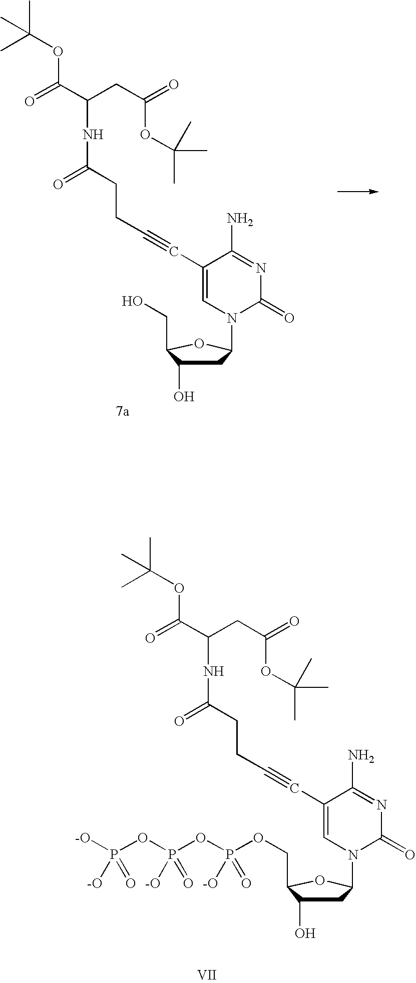

Phosphoroxy chloride (6.0 μl, 0.059 mmol) was added to a cooled solution (0° C.) of 7a (30 mg, 0.054 mmol) in trimethyl phosphate (1 mL). The mixture was stirred for 1 h. A solution of bis-n-tributylammonium pyrophosphate (77 mg, 0.16 mmol) in DMF (1 mL) and tributylamine (40 μl, 0.16 mmol) were added. Water (2 mL) was added pH of the solution was measured to be neutral. The solution was stirred at room temperature for 3 h and at 5° C. overnight. A small amount of compound VII (few mg) was obtained after purification by RP-HPLC (eluent: 100 mM triethylammonium acetate→20% acetonitrile in 100 mM triethylammonium acetate). 7a (18 mg) was regained.

Immediately prior to or subsequent to incorporation the protective di-tert-butyl ester groups may be cleaved to obtain the corresponding free carboxylic acid.

### Examples 34 and 35

### Preparation of the Mononucleotide Building Block (VIII)

### Example 34

### Preparation of 2-Pent-4-ynoylamino-6-(2,2,2-trifluoro-acetylamino)-hexanoic Acid, (8a)

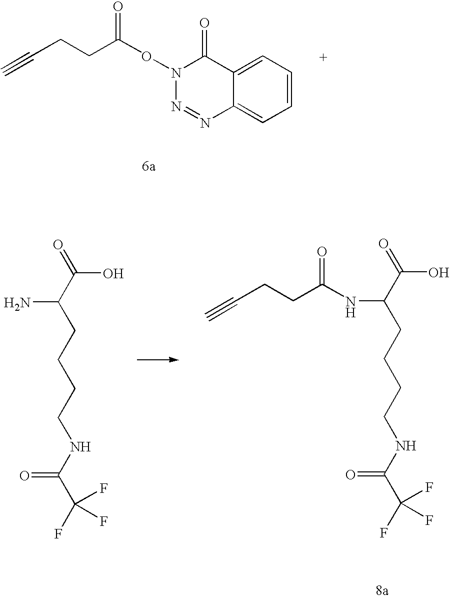

Compound 6a (250 mg, 1.0 mmol) was added to a solution of N—F-trifloroacetyl-L-lysine (Novabiochem, 04-12-5245) (250 mg, 1.0 mmol) in DMF (3 mL). Ethyidiisopropylamine (0.2 mL, 1.2 mmol) was added. The solution was stirred at room temperature overnight and worked-up by RP-HPLC (eluent: water methanol). Yield: 50 mg, 15%. ¹H-NMR (D₂O): δ 4.4 (1H, CH), 3.4 (2H, CH₂), 2.5 (4H, 2×CH₂), 2.3 (1H, CH), 1.9 (1H, CH₂), 1.8 (1H, CH₂) 1.6 (2H, CH₂), 1.5 (2H, CH₂).

### Example 35

### Preparation of 2-{5-[1-(4-Hydroxy-5-(O-triphosphate-hydroxymethyl)-tetrahydrofuran-2-yl)-2,4-dioxo-1,2,3,4-tetrahydro-pyrimidin-5-yl]-pent-4-ynoylamino}-6-(2,2,2-trifluoro-acetylamino)-hexanoic Acid (Compound VIII)

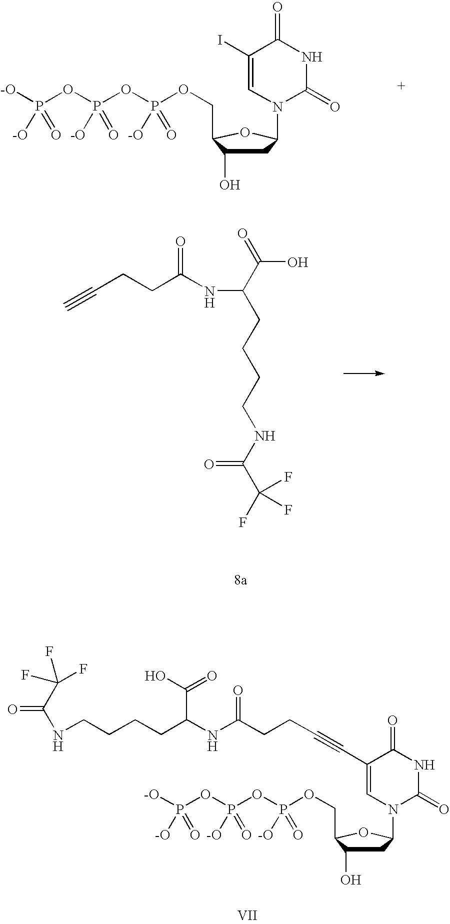

The triethylammonium salt of compound VIII (11 mg) was obtained from compound 8a (50 mg, 0.15 mmol) and 5-Iodo-5′-O-triphosphate-2′-deoxyuridine (50 mg, 0.06 mmol) using the procedure described for the synthesis of compound VI.

### Examples 36 to 40

### Preparation of the Mononucleotide Building Block (IX)

### Example 36

### Preparation of di-Boc-Lysin-propargyl Amide (Compound 9a) C₁₉H₃₃N₃O₅ Mw 383.48

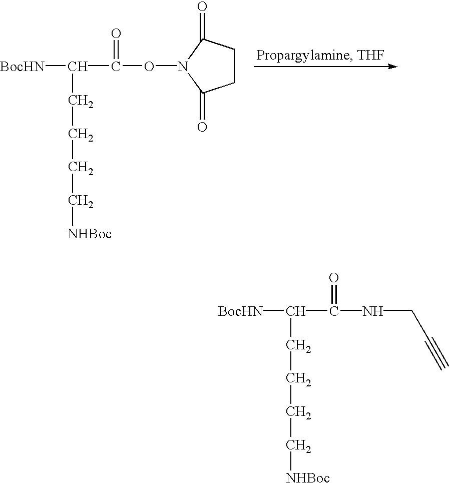

Boc-Lys-(Boc)-OSu (Novabiochem 04-12-0017, 0.887 g, 2 mmol) was dissolved in THF (10 ml). Propargylamine (0.412 ml, 6 mmol) was added and the solution stirred for 2 h. TLC (ethylacetate:heptan 1:1) showed only one product. Dichloromethane (20 ml) was added and the mixture was washed successively with citric acid (1M, 10 ml) and saturated sodium hydrogen carbonate (10 ml). The organic phase was dried with magnesium sulphate filtered and evaporated to give compound 9a (0.730 g) as a colourless syrup.

¹H-NMR: ∂6.55 (1H, NH), 5.15 (1H, NH), 4.6 (1H, CH—NH), 4.05 (2H, CH—C—CH₂—N), 3.75 (1H, NH), 3.1 (2H, CH₂—NH) 2.25 (1H, CH—C—CH₂), 1.9-1.3 (6H, 3×CH₂), 1.4 (18H, 6×CH₃).

### Example 37

### Preparation of 5-Iodo-3′-O-acetyl-5′-O-TBDMS-2′-deoxyuridine (compound 9b) C₁₇H₂₇IN₂O₆Si Mw 510.40

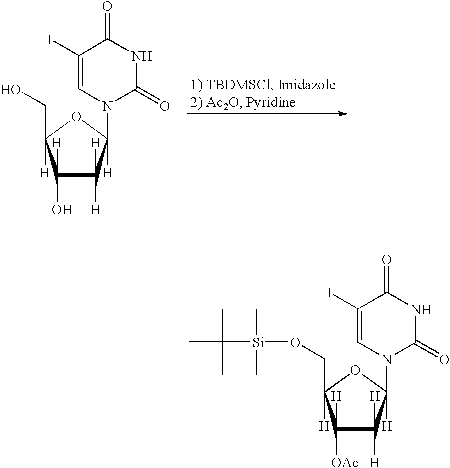

5-Iodo-2′-deoxyuridine (Sigma 1-7125, 2.50 g, 7.06 mmol) and imidazol (0.961 g, 14.12 mmol) was dissolved in DMF (10 ml). Cooled to 0° C. and a solution of TBDMSCI (t-butyl-dimethyl-chloride, 1,12 g, 7.41 mmol) in dichloromethane (5.0 ml) was run in over 20 minutes. Stirring was continued at room temperature for 18 h, and the mixture was evaporated. The crude mono silylated nucleoside was dissolved in pyridine (40 ml) and cooled to 0° C. Acetic anhydride (4.0 ml, 42.3 mmol) was added over 30 minutes and stirring was continued for 18 h at room temperature. The reaction mixture was evaporated and dissolved in dichloromethane (20 ml) and citric acid (2M, 20 ml) was added. The aqueous phase was back extracted with dichloromethane (2×20 ml). The combined organic phases were washed with saturated sodium bicarbonate (20 ml), dried with sodium sulphate and evaporated (5.85 g). Recrystallisation form ethylacetate/EtOH gave 9b (2.54, g) pure for synthesis TLC (Ethyl acetate). Further recrystallisation furnished an analytical pure sample mp.172.4-173.1° C.

### Example 38

### Preparation of 5-Iodo-3′-O-acetyl-2′-deoxyuridine (compound 9c) C₁₁H₁₃1N₂O₆ Mw 396.14

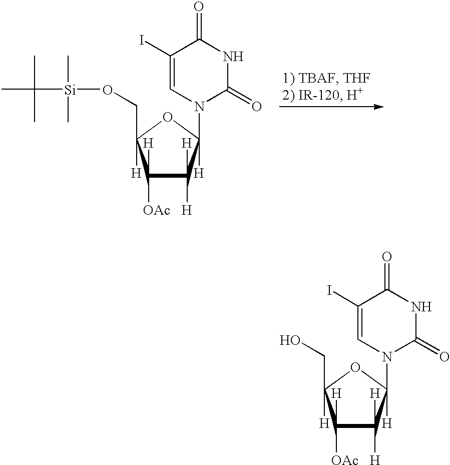

5-Iodo-3′-O-acetyl-5′-O-TBDMS-2′-deoxyuridine (compound 9b) (2.54 g, 4.98 mmol) as dissolved in THF (25 ml), tetra butyl ammonium fluoride trihydrat (TBAF, 3.2 g, 10.1 mmol) was added and stirred for 18 h at room temperature. The reaction mixture was added water (25 ml) stirred for 1 h. Ion exchange resin IR-120H+(26 ml) was then added and stirring was continued for 1 h. The solution was filtered and reduced to approximately 10 ml in vaccuo. Crystals were collected and dried in vaccuo (1.296 g)

### Example 39

### Preparation of 5-Iodo-5′-O-triphosphate-2′-deoxyuridine triethylammonium salt (compound 9d) C₉H₁₄1N₂O₁₄P₃+n·N(CH₂CH₃)₃ Mw 897.61 for n=3.

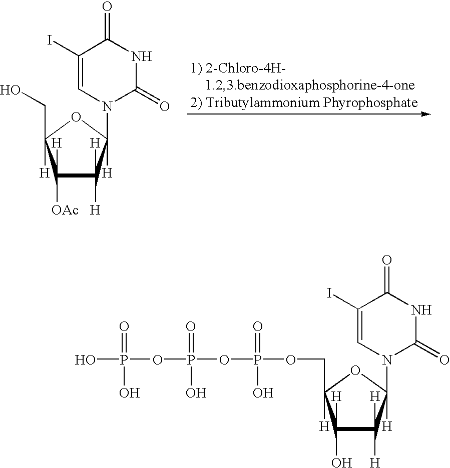

5-Iodo-3′-O-acetyl-2′-deoxyuridine (compound 9c) (2.54 g, 4.98 mmol) as dissolved in pyridine (3.2 ml) and dioxane (10 ml). A solution of 2-chloro-4H-1,3,2-benzodioxaphosphorin-4-one in dioxane (3.60 ml, 1 M, 3.60 mmol) was added under stirring. The reaction mixture was stirred for 10 minutes at room temperature followed by simultaneous addition of bis(tri-n-butylammonium) pyrophosphate in DMF (9.81 ml, 0.5 M, 4.91 mmol) and tri-n-butylamine (3.12 ml, 13.1 mmol). Stirring was continued for 10 minutes and the intermediate was oxidized by adding an iodine solution (90 ml,1% w/v in pyridine/water (98/2, v/v)) until permanent iodine colour. The reaction mixture was left for 15 minutes and then decolourized with sodium thiosulfate (5% aqueous solution, w/v). The reaction mixture was evaporated to yellow oil. The oil was stirred in water (20 ml) for 30 minutes and concentrated aqueous ammonia (100 ml, 25%) was added. This mixture was stirred for 1.5 hour at room temperature and then evaporated to an oil of the crude triphosphate product. The crude material was purified using a DEAE Sephadex A25 column (approximately 100 ml) eluted with a linear gradient of triethyl-ammonium hydrogencarbonate [TEAB] from 0.05 M to 1.0 M (pH approximately 7.0-7.5); flow 8 ml/fraction/15 minutes. The positive fractions were identified by RP18 HPLC eluting with a gradient from 10 mM TEAA (triethylammonium acetate) in water to 10 mM TEAA 20% water in acetonitrile. The appropriate fractions were pooled and evaporated. Yield approximately 1042 mg.

### Example 40

### Preparation of 5-(Lysin-propargyl amide)-5′-triphosphate-2′-deoxycytidine, triethylammonium salt (compound IX) C₁₈H₃₀N₅O₁₅P₃+n·N(CH₂CH₃)₃ Mw 952.95 for n=3

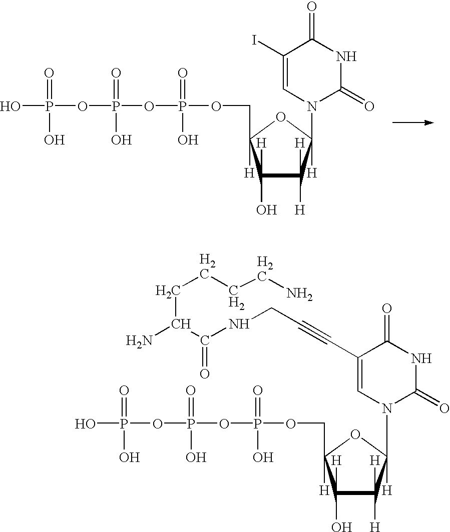

5-Iodo-3′-O-acetyl-5′-triphosphate-2′-deoxyuridine, triethylammonium salt (compound 9d) (0.0087 g, 9.7 μmol) was dissolved in water (100 μl). Air was replaced carefully with argon. Di-Boc-Lysin-propargyl amide (compound 9a) (18.6 mg, 48.5 μmol) dissolved in dioxane (100 μl), triethylamine (2.7 μl, 19.4 μl), Pd((PPh₂)(m-C₆H₄SO₃Na⁺)(H₂O))₄ (compound 9d) (5 mg, 4.4 μmol) and copper (I) iodide (0.4 μl, 2.1 μmol) were added in the given order. The reaction mixture was stirred for 18 h at room temperature in an inert atmosphere then evaporated. The crude material was treated with aqueous hydrochloric acid (0.2 M, 1 ml) for 15 minutes at 30° C. (compound IX) was obtained by HPLC C₁₈ 10 mM TEAA (triethylammonium acetate) in water to 10 mM TEAA 20% water in acetonitrile. Appropriate fractions were desalted using gelfiltration (pharmacia G-10, 0.7 ml).

### Examples 41 to 46

### Preparation of the Mononucleotide Building Block (X)

### Example 41

### Preparation of Boc-Lys-(Boc)-OH (compound 10a) C₁₆H₃₀N₂O₆ Mw 346.42

Lysine (Novabiochem 04-10-0024; 3.65 g, 20 mmol) was dissolved in sodium hydroxide (2 M, 40 ml), added dioxane (60 ml) and di-tert-butyl dicarbonate (8.73 g, 40 mmol) in the given order. The mixture was stirred for 1.75 h at 60° C. Water (50 ml) was added and the solution was washed with dichloromethane (4×25 ml). The aqueous phase was cooled to 0° C. with ice then acidified with 2 M HCl (pH=3) and extracted with dichloromethane (4×25 ml). The organic phase was dried with magnesium sulphate. Evaporation furnished (compound 10a) 6.8 g as a colour less oil.

¹H-NMR: ∂9.5 (1H, COOH), 5.3 (1H, CH), 4.7 (1H, NH), 4.3 (1H, NH), 3.1 (2H, CH₂—NH), 1.8 (2H, CH₂—CH), 1.5(6H, 3×CH₂), 1.45 (18H, 6×CH₃).

### Example 42

### Preparation of di-Boc-Lysin-propargyl Ester (compound 10b) C₁₉H₃₂N₂O₆ Mw 384.47

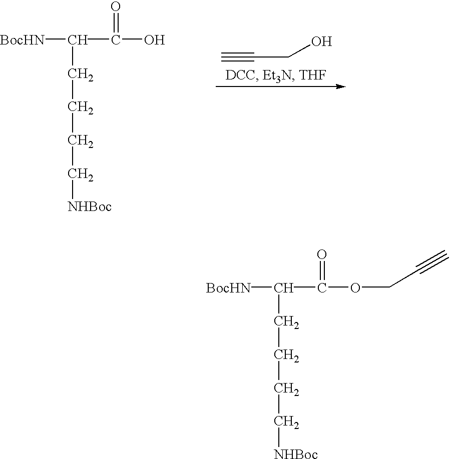

Boc-Lys-(Boc)-OH (compound 10a) (3.46 g, 10 mmol) was dissolved in THF (25 ml). At 0° C. a solution of dicyclohexylcarbodiimide (2.02 g, 10 mmol) in THF (25 ml) and triethylamine (1.39 ml) were added in the given order. The mixture was allowed to warm up to room temperature and stirred for 18 h. The resulting suspension was filtered and evaporated. The oil 5.45 g was pre-purified by column chromatography Heptan: Ethylacetate 3:1.

Pure 10b was achieved by HPLC—C₁₈ 10% MeOH: 90% H₂O→100% MeOH

¹H-NMR: ∂5.1 (1H, NH), 4.75 (2H, CH—C—CH₂—O), 4.6 (1H, NH), 4.35 (1H, CH—NH), 3.1 (2H, CH₂—NH) 2.5 (1H, CH—C—CH₂), 1.9-1.4 (6H, 3×CH₂), 1.5 (18H, 6×CH₃).

### Example 43

### Preparation of 5-Iodo-3′,5′-di-O-TBDMS-2′deoxycytidine (compound 10c) C₂₁H₄₀IN₃O₄Si₂ Mw 581.64

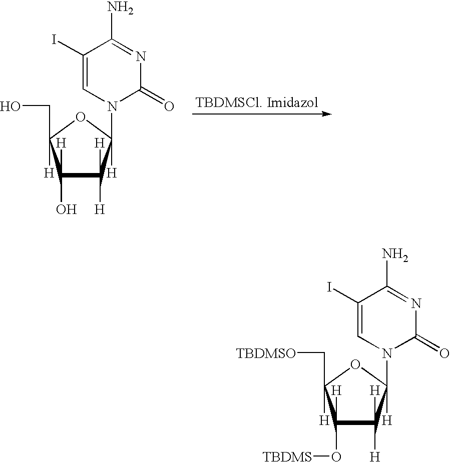

5-Iodo-2-deoxy-Cytidine (Sigma I-7000, 0.353 g, 1 mmol) was dissolved in DMF (4 ml), added t-Butyl-dimethyl silyl chloride (TBDMS-CI, 0.332 g, 2.2 mmol) and Imidazol (0.204 g, 3 mmol). The solution was stirred for 15 h at 50° C. followed by evaporation. Dichloromethane (25 ml) and citric acid (2M, 10 ml) was added to the dry mixture. The aqueous phase was back extracted with dichloromethane (2×10 ml). The combined organic phases were washed with saturated sodium bicarbonate (15 ml), dried with sodium sulphate and evaporated. Compound 10 c (0.405 g) was obtained by recrystallisation from EtOH/Ethylacetate.

¹H-NMR: ∂8.1 (1H, H-6), 6.25 (1H, H-1′), 4.35 (1H, H-4′), 4.0 (1H, H-4′), 3.9 (1H, H-5′), 3.75 (1H, H-5′), 2.5 (1H, H-2′), 1.95 (1H, H-2′), 1.85 (2H, NH), 0.95 (9H, 3×CH₃), 0.9 (9H, 3×CH₃), 0.15 (6H, 2×CH₃), 0.1 (6H, 2×CH₃).

Preparation of 5-(di-Boc-Lysin-propargyl ester)-3′,5′-di-O-TBDMS-2′-deoxycytidine (compound 10d) C₄₀H₇₁IN₅O₁₀Si₂ Mw 838.19

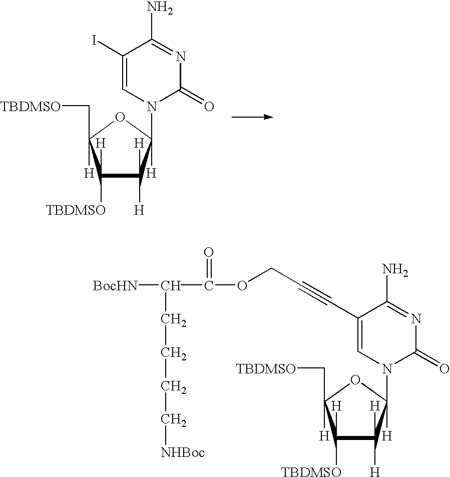

Compound 10c (0.116 g, 0.2 mmol) was dissolved in dichloromethane (10 ml). Air was replaced carefully with argon. Di-Boc-Lysin-propargyl ester (compound 10b) (0.232, 0.6 mmol), triethylamine (0.083 ml, 0.6 mmol), di-chloro-bistriphenylphosphine-palladium 11 (0.0074 g, 0.01 mmol) and copper (I) iodide (0.0038 g, 0.02 mmol) were added in the given order. The reaction mixture was stirred for 15 h at room temperature in an inert atmosphere. The reaction mixture was evaporated re-dissolved in MeOH/H₂O 1:1 1 ml and purified using HPLC-C₁₈ 45% H₂O:55% MeCN→100% MeCN.

¹H-NMR: ∂ ¹H-NMR: ∂ 8.2 (1H, H-6), 6.25 (1H, H-1′), 5.15 (1H, NH), 4.9 (2H, C—CH₂-0), 4.6 (1H, NH), 4.4 (1H, H-4′), 4.3 (1H, CH—NH), 4.0 (1H, H-4′), 3.9 (1H, H-5′), 3.75 (1H, H-5′), 2.5 (1H, H-2′), 3.1 (2H, CH₂—NH), 1.95 (1H, H-2′), 1.9-1.4 (6H, 3×CH₂), 1.85 (2H, NH), 1.5 (18H, 6×CH₃), 0 95 (9H, 3×CH₃), 0.9 (9H, 3×CH₃), 0.15 (6H, 2×CH₃), 0.1 (6H, 2×CH₃).

### Example 44

### Preparation of 5-(di-Boc-Lysin-propargyl ester)-2′-deoxycytidine (compound 10e) C₂₈H₄₃IN₅O₁₀ Mw 609.67

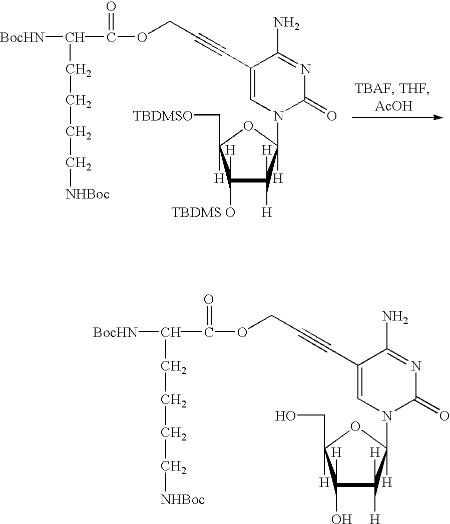

Compound 10d (0.0246 g, 0.029 mmol) was dissolved in THF (1 ml) and successively added acetic acid (0.0165 ml, 0.288 mmol) and tetra n-butyl ammonium fluoride tri-hydrate (0.0454 g, 0.144 mmol). The reaction mixture was stirred for 18 h at room temperature and afterwards evaporated. Re-dissolved in dichloromethane and purified on silica (1×18 cm). Dichloromethane/MeOH 8:2. Fractions which gave UV absorbance on TLC were pooled and evaporated giving 10e (0.0128 g) as a colourless oil.

### Example 45

### Preparation of 5-(Lysin-propargyl ester)-5′-triphosphate-2′-deoxycytidine C₁₈H₃₀N₅O₁₅P₃ Mw 649.38

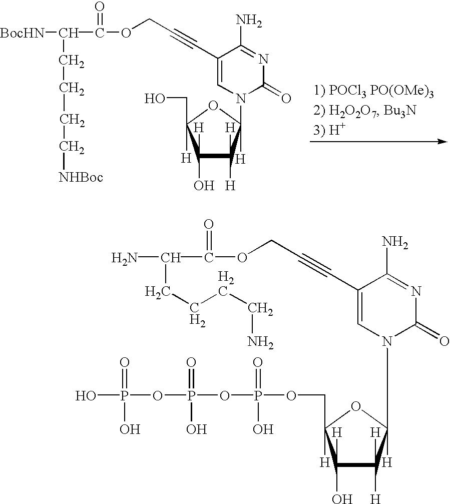

Compound 10e (0.0128 g, 0.021 mmol) was dissolved in trimethylphosphate (0.150 ml) and cooled to ⁰° C. Phosphoroxychloride in trimethylphosphate (1 M, 0.0246 ml) was added slowly in order not to raise the temperature. Stirring was continued for 2 h at 0° C. and the temperature was allowed to rise to ambient. Pyrophosphate in DMF (0.5 M, 0.1025 ml, 0.051 mmol) and tri-n-butyl amine in DMF (1M, 0.0122 ml, 0.051 mmol) were added simultaneous. Stirring was continued for 15 minutes at room temperature and TEAB(triethyl ammonium bicarbonate, 1M, pH=7.3, 0.50 ml) was added. Stirring was continued for 3 h then evaporated.

### Example 46

### Preparation of compound X

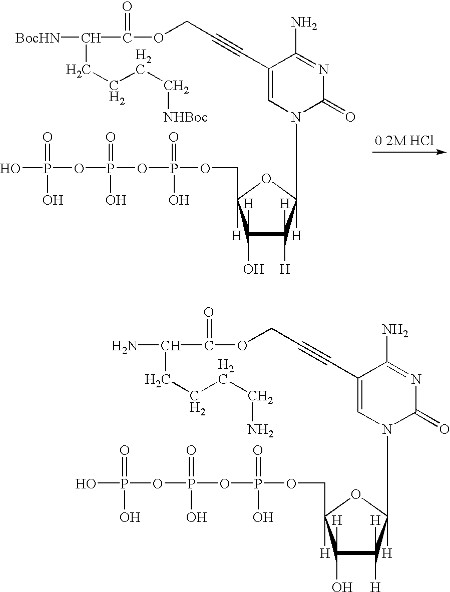

The crude material was treated with aqueous hydrochloric acid (0.2 M, 1 ml) for 15 minutes at 30° C. Compound X was obtained by HPLC C₁₈ 10 mM TEAA (triethylammonium acetate) in water to 10 mM TEAA 20% water in acetonitrile. Appropriate fractions were desalted using gelfiltration (pharmacia G-10, 0.7 ml)

### Example 47

### Polymerase Incorporation of Different Nucleotide Derivatives

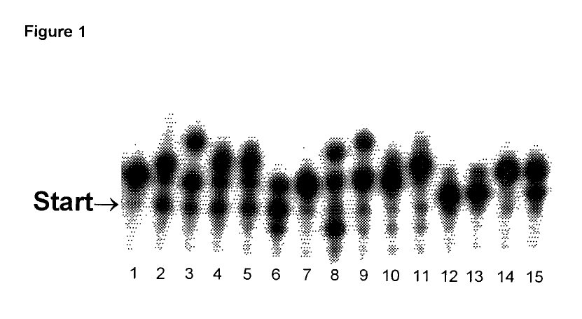

Different extension primers were 5′-labeled with ³²P using T4 polynucleotide kinase using standard protocol (Promega, cat# 4103). These extension primers was annealed to a template primer using 0.1 and 3 pmol respectively in an extension buffer (20 mM Hepes, 40 mM KCl, 8 mM MgCl₂, pH 7.4,10 mM DTT) by heating to 80° C. for 2 min. and then slowly cooling to about 20° C. The wild type nucleotide or nucleotide derivatives was then added (about 100 μM) and incorporated using 5 units AMV Reverse Transcriptase (Promega, part# 9PIM510) at 30° C. for 1 hour. The samples were mixed with formamide dye and run on a 10% urea polyacrylamide gel electrophoresis. The gel was developed using autoradiography (Kodak, BioMax film). The incorporation can be identified by the different mobility shift for the nucleotide derivatives compared to the wild type nucleotide. FIG. 1 shows incorporation of various nucleotide derivates. In lane 1-5 the extension primer 5′-GCT ACT GGC ATC GGT-3′ was used together with the template primer 5′-GCT GTC TGC AAG TGA TAA CCG ATG CCA GTA GC-3′, in lane 6-11 extension primer 5′-GCT ACT GGC ATC GGT-3′ was used together with the template primer 5′-GCT GTC TGC AAG TGA TGA CCG ATG CCA GTA GC-3′, and in lane 12-15 the extension primer 5′-GCT ACT GGC ATC GGT-3′ was used together with the template primer 5′-GCT GTC TGC AAG TGA CGT AAC CGA TGC CAG TAG C-3′. Lane 1, dATP; lane 2, not relevant; lane 3, Compound IX; lane 4, Compound I; lane 5, Compound II; lane 6, no nucleotide; lane 7, dCTP; lane 8, Compound VII; lane 9, Compound X; lane 10, Compound IV; lane 11, Compound III; lane 12, no nucleotide; lane 13, dTTP; lane 14, dTTP and dATP; lane 15, dTTP and Compound X. These results illustrate the possibility to incorporate a variety of nucleotide derivatives of dATP, dTTP and dCTP using different linkers and functional entities. Other polymerases such as Taq, M-MLV and HIV have also been tested with positive results.

The compounds shown in chart 4 may be synthesised by the methods described above.

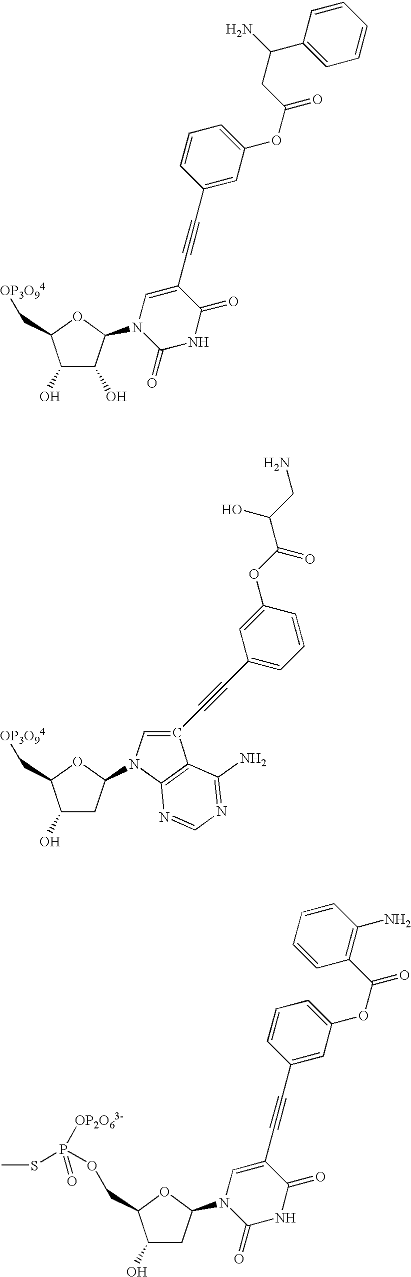

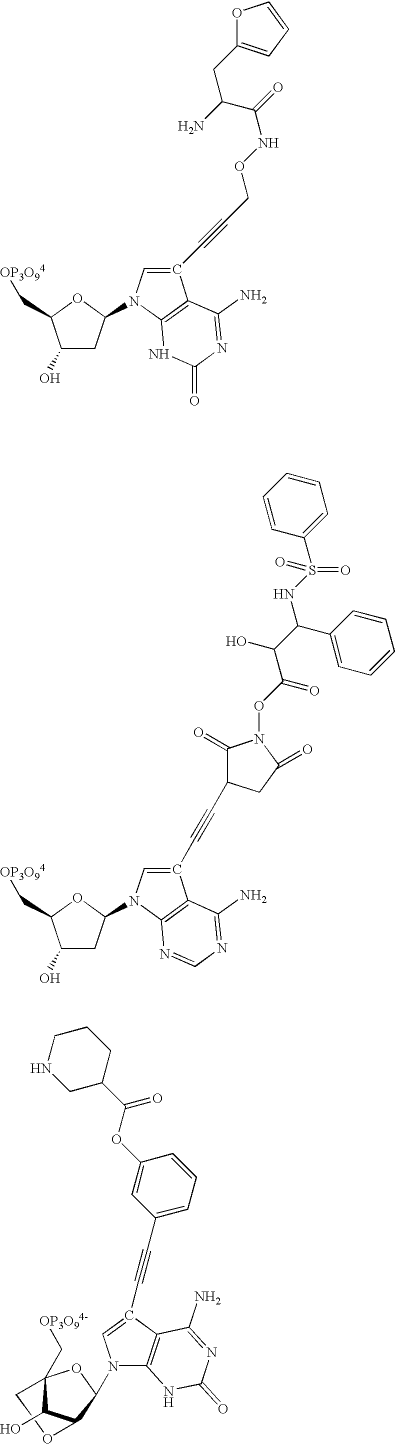

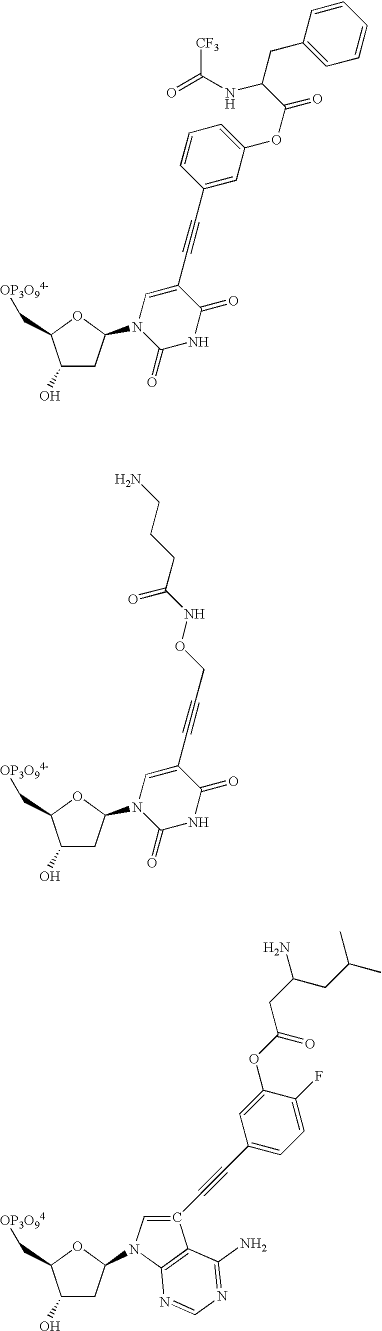

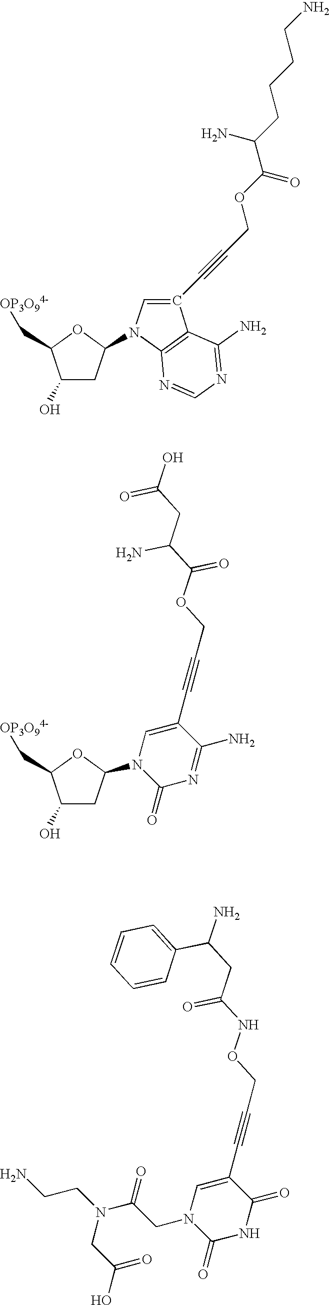

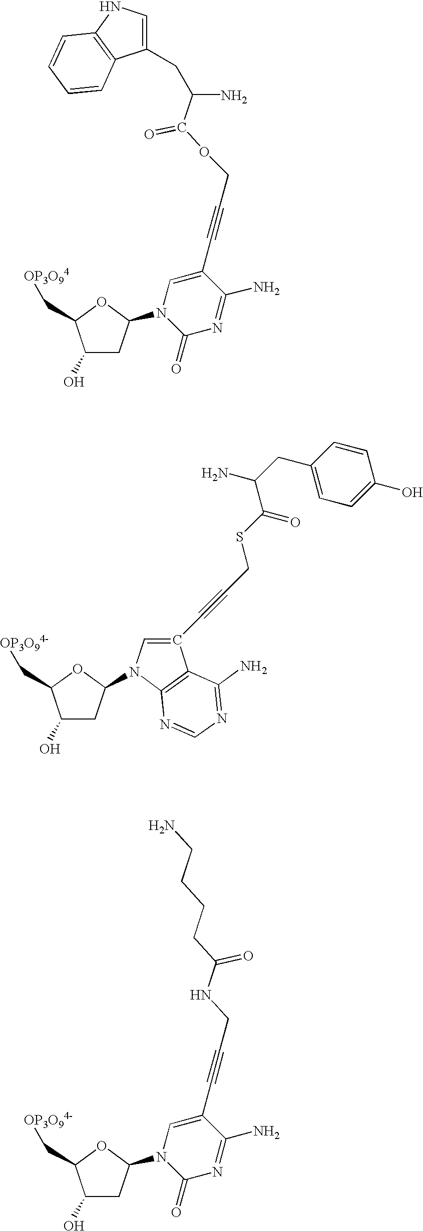

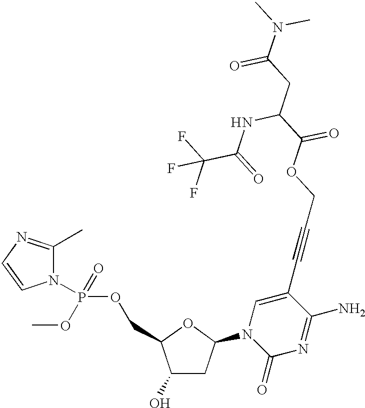

1. A Nucleoside derivative having the general formula: 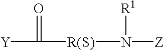
Wherein y is a group 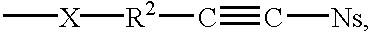
wherein
X is a hetero atom selected from the group O, S, Se or a group NR⁴, wherein R⁴ is hydrogen or an optionally substituted linear or branched C₁₋₆ alkyl or C₂₋₆ alkenyl:
R² is selected from the group consisting of C₁₋₆ alkylen, C₂₋₆ alkylenylen, C₂₋₆ alkynylen, C₃₋₆ cycloalkylen, heterocycloalkylen, —CH₂—O—, arylen or heteroarylen, wherein each of the groups R² are substituted with 0-3 R⁸ groups independently selected from ═O, ═S, —F, —Cl, —Br, —I, —OCH₃, —NO₂ or C₁₋₆ alkyl, and
Ns is a nucleoside analogue consisting of a nucleobase and a backbone unit;
or Y is —OR³, wherein R³ is H or an acid protective group.
R(S) is a C₁₋₁₄ alkylen, C₃₋₁₀ cycloalkylen, aryl, heterocycloalkyl or heteroaryl substituted by n sidechains S, wherein n is an integer of 0 to 4
R¹ is H, C₁₋₆ alkyl substituted with 0-3 R⁹ where R⁹ is independently selected from ═O, Cl, Br, —CN, —OR⁶, —SR⁶, —NR⁶R⁷, —COOR⁶, —CONR⁶R⁷, —SO₂NR⁶R⁷ or a Cl-6 alkylen group forming a ringstructure with S
R⁶ and R⁷ are independently selected from H, C₁₋₆ linear alkyl, C₁₋₆ branched alkyl, C₁₋₆ cycloalkyl, aryl, heteroaryl, aralkyl, or hetero aralkyl.
S is C, 6 linear alkyl, C₃₋₆ branched alkyl, C₃₋₆ cycloalkyl, aryl, heteroaryl, aralkyl, hetero aralkyl substituted with 0-3 R⁵ where R⁵ is independently selected from ═O, Cl, Br, —CN, —OR⁶, —SR⁶, —NR⁶R⁷, —COOR⁶, —CONR⁶R⁷, —SO₂NR⁶R⁷.
Z is H, an amino protective group or a group 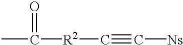
with the proviso, that when Y is not 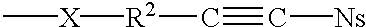
Z is

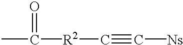

2. A compound according to claim 1 wherein the alkynylen linker is connected to the nucleobase of a nucleoside analogue.

3. A compound according to claim 1 wherein the alkynylen linker is connected to the nucleobase of a nucleoside analogue in the 7 position of the bicyclic purine nucleobases and the 5 position of the monocyclic pyrimidine bases.

4. A compound according to any of the claims 1, or 2-3 wherein R² is selected from the group consisting of C₁₋₆ alkylen, C₂₋₆ alkylenylen, C₂₋₆ alkynylen, heterocycloalkylen, —CH₂—O—, arylen or heteroarylen, wherein each of the groups R² are substituted with 0-3 R⁸ groups independently selected from ═O, —F, —Cl, —Br, —NO₂, C₁₋₆ alkyl.

5. A compound according to any of the claims 1, or 2-3 wherein R² is selected from the group consisting of C₁₋₆ alkylen, C₂₋₆ alkynylen, heterocycloalkylen, —CH₂—O—, arylen or heteroarylen, wherein each of the groups R² are substituted with 0-2 R⁸ groups independently selected from ═O, —F, —NO₂, C₁₋₆ alkyl.

6. A compound according to any of the claims 1, or 2-3 wherein R² is selected from the group consisting of —CH₂—, —CH₂CH₂—, 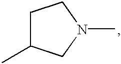
, —CH₂—O—, or arylen wherein each of the groups R² are substituted with 0-2 R⁸ groups independently selected from ═O, —F, —NO₂, C₁₋₆ alkyl.

7. A compound according to any of the claims 1, or 2-3 wherein R² is selected from the group consisting of —CH₂—, —CH₂CH₂—, 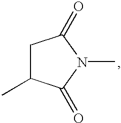
, —CH₂—O—, or arylen.

8. A compound according to any of the claims 1, or 2-3 wherein R² is selected from the group consisting of —CH₂—, —CH₂CH₂—, 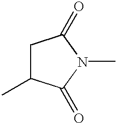
or arylen.

9. A compound according to any of the claims 1, 2-3 or 4-8 wherein X is O

10. A compound according to any of the claims 1, 2-3 or 4-8 wherein X is S

11. A compound according to any of the claims 1, 2-3 or 4-8 wherein X is NR⁴

12. A compound according to any of the claims 1, 2-3 or 4-8 wherein X is NR⁴ and R⁴ is H or —CH₃

13. A compound according to any of the claims 1, 2-3 or 4-8 wherein X is NH

14. A compound according to any of the claims 1, 2-3, 4-8 or 9, 10 or 11-13 wherein R(S) is a C₁₋₄ alkylene, C₃₋₁₀ cycloalkylen, aryl, heterocycloalkyl or heteroaryl substituted by n sidechains S, wherein n is an integer of 0 to 3

15. A compound according to any of the claims 1, 2-3, 4-8 or 9,10 or 11-13 wherein R(S) is a C₁₋₄ alkylene, aryl or heteroaryl substituted by n sidechains S, wherein n is an integer of 0 to 3

16. A compound according to any of the claims 1, 2-3, 4-8 or 9, 10 or 11-13 wherein R(S) is a C₁₋₄ alkylene substituted by n sidechains S, wherein n is an integer of 0 to 3

17. A compound according to any of the claims 1, 2-3, 4-8 or 9, 10 or 11-13 wherein R(S) is a C₁₋₂ alkylene substituted by n sidechains S, wherein n is an integer of 0 to 3

18. A compound according to any of the claims 1, 2-3, 4-8 or 9, 10 or 11-13 wherein R(S) is a C₁₋₂ alkylene substituted by n sidechains S, wherein n is an integer of 0 to 2

19. A compound according to any of the claims 1, 2-3, 4-8 or 9, 10 or 11-13 wherein R(S) is a C₁₋₂ alkylene substituted by n sidechains S, wherein n is an integer of 0 to 1

20. A compound according to any of the claims 1, 2-3, 4-8, 9, 10, 11-13 or 14-19 wherein S is C₁₋₆ linear alkyl, C₃₋₆ branched alkyl, C₃₋₆ cycloalkyl, aryl, heteroaryl, aralkyl, hetero aralkyl substituted with 0-3 R⁵ where R⁵ is independently selected from ═O, Cl, Br, —CN, —OR⁶, —SR⁶, —NR⁶R⁷, —COOR⁶, —CONR⁶R⁷, —SO₂NR⁶R⁷ where R⁶ and R⁷ are independently selected from H, C₁₋₃ linear alkyl, C₃₋₆ cycloalkyl, aryl, heteroaryl, aralkyl, or hetero aralkyl.

21. A compound according to any of the claims 1, 2-3, 4-8, 9, 10, 11-13 or 14-19 wherein S is C₁₋₆ linear alkyl, C₃₋₆ branched alkyl, C₃₋₆ cycloalkyl, aryl, heteroaryl, aralkyl, hetero aralkyl substituted with 0-2 R⁵ where R⁵ is independently selected from ═O, Cl —CN, —OR⁶ SR⁶ NR⁶R⁷—COOR⁶—CONR⁶R⁷—SO₂NR⁶R⁷ where R⁶ and R⁷ are independently selected from H, C₁₃ linear alkyl, aryl, heteroaryl, aralkyl, or hetero aralkyl.

22. A compound according to any of the claims 1, 2-3, 4-8, 9, 10, 11-13 or 14-19 wherein S is Cl₆ linear alkyl, C₃₋₆ branched alkyl, C₃₋₆ cycloalkyl, aryl, heteroaryl, aralkyl, hetero aralkyl substituted with 0-2 R⁵ where R⁵ is independently selected from ═O, Cl, —CN, —OR⁶ SR⁶ NR⁶R⁷—COOR⁶—CONR⁶R⁷—SO₂NR⁶R⁷ where R and R⁷ are independently selected from H and C₁₋₃ linear alkyl

23. A compound according to any of the claims 1, 2-3, 4-8, 9, 10, 11-13 or 14-19 wherein S is C₁₋₆ linear alkyl, C₃₋₆ branched alkyl, C₃₋₆ cycloalkyl, aryl, heteroaryl, aralkyl, hetero aralkyl substituted with 0-1 R⁵ where R⁵ is selected from ═O, Cl, —CN, —OR⁶, —SR⁶, —NR⁶R⁷, —COOR⁶, —CONR⁶R⁷, —SO₂NR⁶R⁷ where R⁶ and R⁷ are independently selected from H and C₁₋₃ linear alkyl

24. A compound according to any of the claims 1, 2-3, 4-8, 9, 10, 11-13 or 14-19 wherein S is C₁₋₆ linear alkyl or aryl substituted with 0-1 R⁵ where R⁵ is selected from ═O, Cl, —CN, —OR⁶, —SR⁶, —NR⁶R⁷, —COOR⁶, —CONR⁶R⁷, —SO₂NR⁶R⁷ where R⁶ and R⁷ are independently selected from H and C₁₋₃ linear alkyl

25. A compound according to any of the claims 1, 2-3, 4-8, 9, 10, 11-13 or 14-19 wherein S is C₁₋₆ linear alkyl or aryl.

26. A compound according to any of the claims 1, 2-3, 4-8, 9, 10, 11-13, 14-19 or 20-25 wherein R¹ is H, C₁₋₆ alkyl substituted with 0-1 R⁹ where R⁹ is independently selected from ═O, Cl, Br, —CN, —OR⁶, —SR⁶, —NR⁶R⁷, —COOR⁶, —CONR⁶R⁷, —SO₂NR⁶R⁷ wherein R⁶ and R⁷ are independently selected from H, Cl-₆ linear alkyl, C₁₋₆ branched alkyl, C₁₋₆ cycloalkyl, aryl, heteroaryl, aralkyl, or hetero aralkyl or a C₁₋₆ alkylen group forming a ringstructure with S.

27. A compound according to any of the claims 1, 2-3, 4-8, 9, 10, 11-13, 14-19 or 20-25 wherein R¹ is H, C₁₋₆ alkyl or a C₁₋₆ alkylen group forming a ringstructure with S

28. A compound according to any of the claims 1, 2-3, 4-8, 9, 10, 11-13, 14-19 or 20-25 wherein R¹ is H or a C₁₋₆ alkylen group forming a ringstructure with S.

29. A compound according to any of the claims 1, 2-3, 4-8, 9, 10, 11-13, 14-19 or 20-25 wherein R¹ is H or C₁₋₆ alkyl.

30. A compound according to any of the claims 1, 2-3, 4-8, 9, 10, 11-13, 14-19 or 20-25 wherein R¹ is H.

31. A compound according to any of the claims 1, 2-3, 4-8, 9, 10, 11-13, 14-19, 20-25 or 26-30 wherein Z is H, an amino protective group selected from the group of formyl, acetyl, trifluoroacetyl, benzoyl, tert-butyloxycarbonyl, triphenylmethyl, benzyl, benzyloxycarbonyl or tosyl or a group 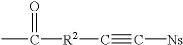
with the proviso, that when Y is not 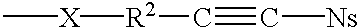
is

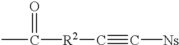

32. A compound according to any of the claims 1, 2-3, 4-8, 9, 10, 11-13, 14-19, 20-25 or 26-30 wherein Z is H, an amino protective group selected from the group of acetyl, trifluoroacetyl, tert-butyloxycarbonyl or tosyl or a group 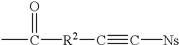
with the proviso, that when Y is not 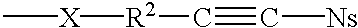
is

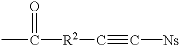

33. A compound according to any of the claims 1, 2-3, 4-8, 9, 10, 11-13, 14-19, 20-25, 26-30 or 31-32 wherein the nucleobase is uracil or cytosine modified in the 5 position or 7-adeazaadenine or 7-deazaguanidine modified in the 7 position.

34. A compound according to any of the claims 1, 2-3, 4-8, 9, 10, 11-13, 14-19, 20-25, 26-30, 31-32 or 33 wherein the backbone unit type is DNA, RNA, Oxy-LNA, Thio-LNA, Amino-LNA, Phosphorthioate, 2′-O-methyl, PNA or Morpholino as described in chart 3.

35. A compound according to any of the claims 1, 2-3, 4-8, 9, 10, 11-13, 14-19, 20-25, 26-30, 31-32 or 33 wherein the backbone unit type is DNA, RNA, Oxy-LNA, PNA or Morpholino

36. A compound according to any of the claims 1, 2-3, 4-8, 9, 10, 11-13, 14-19, 20-25, 26-30, 31-32 or 33 wherein the backbone unit type is DNA, PNA or Oxy-LNA

37. A compound according to any of the claims 1, 2-3, 4-8, 9, 10, 11-13, 14-19, 20-25, 26-30, 31-32 or 33 wherein the backbone unit type is DNA

38. A compound according to any of the claims 1, 2-3, 4-8, 9, 10, 11-13, 14-19, 20-25, 26-30, 31-32 or 33 wherein the backbone unit type is Oxy-LNA

39. A compound according to any of the claims 1, 2-3, 4-8, 9, 10, 11-13, 14-19, 20-25, 26-30, 31-32 or 33 wherein the backbone unit type is PNA

40. A compound according to any of the claims 1, 2-3, 4-8, 9, 10, 11-13, 14-19, 20-25, 26-30, 31-32, 33 or 34-39 wherein more nucleoside analogues are connected via their backbone structures forming di-, tri- or oligomeric nucleoside analogues as building blocks

41. A compound according to any of the claims 1, 2-3, 4-8, 9, 10, 11-13, 14-19, 20-25, 26-30, 31-32, 33, 34-39 or 40 wherein Y is 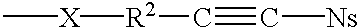
or —OR³ wherein R³ is selected from the group H, C₁₋₃ alkyl, allyl, benzyl, tert-butyl or triphenylmethyl.
